(19) United States
(12) Patent Application Publication
(10) Pub. No.: US 2026/0262968 A1
(43) Pub. Date: Sep. 10, 2026

(54) GROOVED MICRONEEDLES FOR PASSIVE AND ACTIVE SAMPLING OF INTERSTITIAL FLUIDS

(71) Applicant: Trustees of Tufts College, Medford, MA (US)

(72) Inventor: Sameer Sonkusale, Medford, MA (US)

(21) Appl. No.: 19/166,982

(22) PCT Filed: Mar. 21, 2024
(86) PCT No.: PCT/US2024/020874

§ 371 Date: Sep. 19, 2025

Related U.S. Application Data
(60) Provisional application No. 63/491,521, filed on Mar. 21, 2023.

(51) Int. Cl. A61B 5/145; A61B 5/00; A61B 10/00

(52) U.S. Cl. CPC A61B 5/14514; A61B 5/6833; A61B 5/685; A61B 10/0045; A61B 2010/0006; A61B 2010/008; A61B 2560/0443

(57) ABSTRACT

Disclosed are systems and methods for sampling a fluid from a region of interest of a subject. The sampling system includes a patch including at least one grooved microneedle coupled thereto, a collection reservoir coupled to the patch, and a sensor coupled to the collection reservoir. The at least one grooved microneedle defines a fluid channel therein, and the collection reservoir is in fluid communication with the fluid channel of the at least one grooved microneedle.

### CROSS-REFERENCE TO RELATED APPLICATIONS

This application claims priority to and the benefit of U.S. Provisional Patent Application No. 63/491,521, filed on Mar. 21, 2023, which is incorporated herein by reference in its entirety.

### STATEMENT REGARDING FEDERALLY FUNDED RESEARCH

Not applicable.

### BACKGROUND

Skin is considered as the largest organ in the body with around 1.5 m² surface area in adults and contains a wide variety of biomarkers that can be analyzed to determine metabolic function characteristics of a subject. However, the outermost layer of skin, stratum corneum, which protects the human body from toxic chemicals, makes it challenging for non-invasive sampling of these biomarkers to provide reliable information over sustained sampling periods. Moreover, typical sampling techniques, such as the withdrawal of fluid from a subject using hypodermic needles, can be uncomfortable and/or painful for the subject, and such techniques are often difficult to master given the anatomical variability between different subjects.

In recent years, there have been attempts to utilize microneedles to provide a painless and non-invasive method of sampling interstitial fluid due to their hygienic qualities and low disposal cost. Specifically, microneedles create microchannels in the skin, which allow for easy fluid transport. However, traditional microneedles have generally fallen into four classifications: solid, coated, dissolving, and hollow. Meanwhile, the traditional materials used to create microneedles can generally be divided into two main categories, namely, microneedles comprising soft materials, i.e., having elastic moduli close to that of skin, and microneedles comprising hard materials, i.e., having elastic moduli much larger than that of skin.

Hard microneedles have much higher Young's moduli when compared to skin, and therefore can effectively penetrate into different skin types. However, recent attempts at fabricating hard, hollow microneedles for ISF sampling have resulted in low-resolution molds that lack the capillary action structures, such as adequate sampling surface area, which are critical for effective fluid sampling. Therefore, there exists a need for systems and methods of using and manufacturing microneedles with improved fluid sampling characteristics, while also retaining critical structural properties that aid in overcoming the obstacles associated with traditional fluid sampling techniques, as discussed above.

### SUMMARY

The present disclosure overcomes the aforementioned drawbacks by providing systems and methods for creating efficient, compact microneedle arrays capable of effectively sampling interstitial fluid at a surface region of interest of a subject. The systems and methods provided herein can be achieved in a cost- and time-efficient manner compared to traditional systems and methods.

In accordance with some aspects of the present disclosure, a sampling system includes a patch including at least one grooved microneedle coupled thereto, a collection reservoir coupled to the patch, and a sensor coupled to the collection reservoir. The at least one grooved microneedle defines a fluid channel therein, and the collection reservoir is in fluid communication with the fluid channel of the at least one grooved microneedle.

In accordance with some aspects of the present disclosure, a system for sampling a fluid of a subject includes a patch including at least one grooved microneedle coupled thereto and a sensor to detect a presence of at least one biomarker in the fluid that is drawn through the at least one microneedle. The at least one grooved microneedle defines a fluid channel therein, and the collection reservoir is in fluid communication with the fluid channel of the at least one grooved microneedle.

In accordance with some aspects of the present disclosure, a method of sampling interstitial fluid comprises placing, on a subject's skin, a microneedle patch including at least one microneedle coupled to a first side of the microneedle patch and a collection reservoir coupled to a second side of the microneedle patch. The method further includes providing a suction force to the microneedle patch to direct a fluid through the at least one microneedle and into the collection reservoir. The method further includes detecting, with a sensor coupled to the collection reservoir, at least one of a presence or a concentration of a biomarker in the fluid.

The foregoing and other advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1A is a schematic diagram of an example sampling system including absorbent beads in an unexpanded state, in accordance with some aspects of the present disclosure.

FIG. 1B is a schematic diagram of the absorbent beads shown in FIG. 1A in an expanded state.

FIG. 2A is a schematic diagram of another example sampling system including grooved microneedles and a passive suction device in a compressed state, in accordance with some aspects of the present disclosure.

FIG. 2B is a schematic diagram of the example sampling system of FIG. 2A with the passive suction device in an expanded state.

FIG. 3A is a perspective view of a grooved microneedle array used for interstitial fluid extraction including an absorbent bead in an initial non-expanded state, in accordance with some aspects of the present disclosure.

FIG. 3B is a perspective view of the microneedle array of FIG. 3A with the absorbent bead in an intermediate sampling state.

FIG. 3C is a perspective view of the microneedle array of FIG. 3A with the absorbent bead in a fully expanded state.

FIG. 4 is a schematic diagram of yet another sampling system including an active pressure syringe pump, in accordance with some aspects of the present disclosure.

FIG. 5 is a schematic diagram of still another example sampling system including an active pressure micropump, in accordance with some aspects of the present disclosure.

FIG. 6 is a schematic diagram of a detection system configured to detect one or more biomarkers in a fluid, in accordance with some aspects of the present disclosure.

FIG. 7 is a flowchart of non-limiting example steps for a method of sampling interstitial fluid, in accordance with some aspects of the present disclosure.

FIG. 8A is a rear side view of an example grooved microneedle of a microneedle array, in accordance with some aspects of the present disclosure.

FIG. 8B is a front side view of the example grooved microneedle of FIG. 8A.

FIG. 8C is a top plan view of the example grooved microneedle of FIG. 8A.

FIG. 8D is an isometric view the example grooved microneedle of FIG. 8A.

FIG. 8E is a perspective view of a molded microneedle that is manufactured based on the design of the example grooved microneedle of FIG. 8A.

FIG. 8F is a perspective view of an example grooved microneedle array, in accordance with some aspects of the present disclosure.

FIG. 8G is a perspective view of a molded microneedle array that is manufactured based on the design of the example grooved microneedle array of FIG. 8F.

FIG. 9A is an isometric view of another example grooved microneedle including a central channel, in accordance with some aspects of the present disclosure.

FIG. 9B is a perspective view of a molded microneedle that is manufactured based on the design of the example grooved microneedle of FIG. 9A.

FIG. 9C is a top view of another example molded microneedle array including microneedles similar to the molded microneedle of FIG. 9B.

FIG. 9D is a detail perspective view of the example molded microneedle array of FIG. 9C.

FIG. 10 is a preparation scheme of a side cross-sectional view of a grooved microneedle array, in accordance with some aspects of the present disclosure.

FIG. 11A is a series of images of a fluid extraction behavior of a grooved microneedle array including expandable absorbent beads, in accordance with some aspects of the present disclosure.

FIG. 11B is a plot illustrating a comparison of changes in height and width over time of the absorbent bead of FIG. 11A during fluid sampling.

FIG. 12A is an image of a top view of a grooved microneedle array with a channel width of 50 μm, in accordance with some aspects of the present disclosure.

FIG. 12B is an image of a top view of a grooved microneedle array with a channel width of 100 μm, in accordance with some aspects of the present disclosure.

FIG. 12C is an image of a top view of a grooved microneedle array with a channel width of 200 μm, in accordance with some aspects of the present disclosure.

FIG. 12D is an image of a side view of the grooved microneedle array of FIG. 12A.

FIG. 12E is an image of a side view of the grooved microneedle array of FIG. 12B.

FIG. 12F is a side view of an image of the grooved microneedle array of FIG. 12C.

FIG. 13A is an image of a side view of a 300 μm inner diameter microneedle array coupled to a fluid-sampling port, in accordance with some aspects of the present disclosure.

FIG. 13B is an image of a top view of the microneedle array of FIG. 13A.

FIG. 13C is an image of a side perspective view of the microneedle array of FIG. 13A.

FIG. 14A is a top view of a puncture pattern formed by inserting a microneedle array into a gel, in accordance with some aspects of the present disclosure.

FIG. 14B is a top view of the microneedle array of FIG. 14A after passively sampling sulforhodamine B dye solution therewith.

FIG. 14C is an image of an experimental setup including the microneedle array of FIG. 14A before actively sampling sulforhodamine B dye solution with a piezo pump.

FIG. 14D is an image of the experimental setup of FIG. 14C after actively sampling the sulforhodamine B dye solution with the piezo pump.

FIG. 15A is a schematic diagram of yet another example microneedle with an angled body, in accordance with some aspects of the present disclosure.

FIG. 15B is a detail left side view of a microneedle array incorporating the design of the example microneedle of FIG. 15A.

FIG. 15C is a detail top view of the microneedle array of FIG. 15B.

FIG. 15D is a rear side view of the microneedle array of FIG. 15B.

FIG. 16 is a schematic diagram of another example sampling system including a handheld vacuum pump, in accordance with some aspects of the present disclosure.

FIG. 17A is an image of a reservoir of a microneedle sampling system before sampling occurs, in accordance with some aspects of the present disclosure.

FIG. 17B is an image of the reservoir of FIG. 17A after being partially filled with sampled solution, in accordance with some aspects of the present disclosure.

FIG. 17C is an image of the reservoir of FIG. 17A after being completely filled with sampled solution, in accordance with some aspects of the present disclosure.

FIG. 18A is an image of a front view of a microneedle patch, in accordance with some aspects of the present disclosure.

FIG. 18B is an image of a rear view of the microneedle patch of FIG. 18A.

FIG. 18C is an image of a sensor that is configured to be coupled to the microneedle patch of FIG. 18A.

FIGS. 19A-19D are a series of images of a method of detecting a presence and/or a concentration of a biomarker with a paper-based sensor, in accordance with some aspects of the present disclosure.

FIG. 20A-20F are a series of images of the paper-based sensor of FIGS. 19A-19C depicting colorimetric changes based on fluid sample volume.

FIG. 21A is a plot illustrating an effect of pH of a citate buffer solution on a colorimetric change distance in a sensor, in accordance with some aspects of the present disclosure.

FIG. 21B is a plot illustrating an effect of concentration of a citate buffer solution on a colorimetric change distance in a sensor, in accordance with some aspects of the present disclosure.

FIG. 21C is a plot illustrating an effect of concentration of a bromothymol blue molecule on a colorimetric change distance in a sensor, in accordance with some aspects of the present disclosure.

### DETAILED DESCRIPTION

Before the present invention is described in further detail, it is to be understood that the invention is not limited to the particular embodiments described. It is also understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a controller device, a process being executed (or executable) by a controller device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other controller devices, or may be included within another component (or system, module, and so on).

In the methods described herein, the steps can be carried out in any order without departing from the principles of the disclosure, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated.

Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process. The term “substantially” as used herein refers to a majority of, or mostly, as in at least about 50%, at least about 60%, at least about 70%, at least about 80%, at least about 90%, at least about 95%, at least about 96%, at least about 97%, at least about 98%, at least about 99%, at least about 99.5%, at least about 99.9%, at least about 99.99%, or at least about 99.999% or more.

The following discussion is presented to enable a person skilled in the art to make and use aspects of the disclosure. Various modifications to the illustrated configurations or processes will be readily apparent to those skilled in the art, and the generic principles herein can be applied to other aspects and applications within the scope of the present disclosure and the understanding of one of skill based thereon. Thus, the present disclosure is not intended to be limited to particular embodiments or aspects shown, but is to be accorded the widest scope consistent with the principles and features disclosed herein. The following detailed description is to be read with reference to the figures, in which like components or elements in different figures have like reference numerals. The figures, which are not necessarily to scale, depict selected aspects and configurations or processes and are not intended to limit the scope of the disclosure. Skilled artisans will recognize the examples provided herein have many useful alternatives and fall within the scope of the disclosure. In accordance with aspects of the present disclosure, mechanisms (which can, for example, include systems, methods, and media) for using sampling systems with grooved microneedles to sample a fluid, e.g., ISF, of a subject are provided.

Generally, the present disclosure provides systems, methods, and media for using a sampling system with at least one microneedle to advantageously place a microneedle patch on a subject's skin, draw fluid through the at least one microneedle from a region of interest of a subject, and analyze the drawn fluid, e.g., to determine the presence and/or concentration of a biomarker in the fluid. To accomplish this, a microneedle can define a fluid path therein, e.g., a groove extending along a length of a microneedle measured between a pointed apex and a base thereof. When the one or more of the microneedles is applied to the skin of a subject, a fluid can be drawn upward through the grooves of the one or more microneedles via capillary action of the grooves. The fluid can then be analyzed using a sensor or sensors. For example, the fluid can be collected in a collection reservoir that is in fluid communication with the one or more microneedles, and the collection reservoir can be coupled to a sensor to perform downstream analysis for a variety of different biomarkers and functions. Thus, one of several features that distinguishes the present disclosure is that a microneedles can define a fluid channel therein and can provide for passive or active sampling of a fluid, e.g., ISF, when applied to the skin of a subject. Moreover, using a grooved microneedle array, e.g., an array disposed on a patch, can increase sampling area and ensure multiple points of contact with the surrounding ISF in a region of interest of a subject. In addition, the sampling systems and microneedles discussed herein and are much easier to fabricate than traditional microneedles by using more efficient molding techniques. Further, the sampling systems discussed herein can be used to sample a fluid of a subject in a variety different ways such as, for example, passively sampling ISF via capillary action of fluid channels in grooved microneedles, actively sampling ISF via negative pressure loading using a flexible membrane, and/or actively sampling ISF using micropumps, handheld pumps, syringe pumps, or another active sampling method.

The inventive microneedles also possess remarkable physical characteristics that confer practical benefits. The dimensions of the grooves of the microneedles can be modulated to change the microneedles' sampling characteristics, e.g., sampling rate. The composition and structure of the microneedle provide excellent penetration into skin as well as structural integrity, e.g., rigidity or semi-rigidity and resistance to breakage.

### Overview of Microneedle Structure and Fabrication

In some embodiments, the system includes a plurality of grooved microneedles, each microneedle comprising a solid material in a conical shape with a groove, which extends along a length of the microneedle between a distal pointed apex and a base. Further, the groove defines a fluid channel along a length of the body of the microneedle. While the figures and examples herein illustrate the groove extending along the entire length of the microneedle between the distal pointed apex and the base, thus defining a longest length of the groove in some aspects, the term “length” is to be construed as a length taken at any point along each respective element of the microneedle.

In some embodiments, the solid material is formed from a flowable material, e.g., a resin that is later cured to form a solid, e.g., a polymer; or a flowable metal, e.g., an alloy, that is later cooled to form a solid. In some embodiments, the flowable material is cast onto a mold comprising one or more needle-shaped mold cavities. Solidification of the flowable material in the mold yields the inventive microneedle(s). In some embodiments, the solid material has a hardness of at least 40 Shore A, between 40 Shore A and 100 Shore A, between 60 Shore A and 100 Shore A, between 0 Shore D and 90 Shore D, 10 Shore D and 80 Shore D, 40 Shore D and 80 Shore D, 60 Shore A and 80 Shore D, or at least 80 Shore D.

In some embodiments, the mold comprises an array of needle-shaped mold cavities in a specific geometric configuration. In some embodiments, the flowable material may be a biocompatible resin. For example, dental SG resin may be used. Other suitable types of biocompatible resins include, but are not limited to, BioMed Clear Resin (RS-F2-BMCL-01), Biomed Amber Resin (RS-F2_BMAM-01), Dental LT Clear Resin (RS-F2-DLCL-02), Surgical Guide Resin (RS-F2-SGAM-01), and Dental SG resin (RS-F2-DGOR-01). The biocompatible resin may be photo-curable and, when cured, may yield a hard polymer. In some embodiments, the biocompatible resin or its cured product may include a species selected from chitosan, chitosan polybutylene adipate terephthalate, poly(butylene adipate-co-terephthalate), polyethylene glycol, poly(ethylene glycol) diacrylate, gelatin, gelatin methacyloyl, polyvinyl alcohol, silk, and combinations thereof. Other materials used to fabricate the porous microneedles may include, but are not limited to, polylactic acid (PLA), polyvinyl alcohol (PVA), poly(ethylene glycol diacrylate) (PEGDA), or UV curable polymers.

### Back Substrate

The material attached to the base(s) of one or more microneedles for supportive purposes, e.g., to provide an adhesive backing and/or to arrange multiple microneedles in an array, is herein referred to as a back substrate. It is contemplated that the back substrate is coupled to a patch, e.g., a microneedle patch, or the back substrate is integral with the patch. In some embodiments, the back substrate can be, or can comprise, a thin elastic, a flexible adhesive, a woven material, a film, a bandage or dressing, a biodegradable material, or any combination thereof. In some embodiments, the back substrate can act as an intermediate adhesive to a larger patch for clinical application. In some embodiments the back substrate can be a continuation of the same material comprising the microneedles. In some embodiments the back substrate can be a secondary, drug-loaded material, including a drug-loaded porous material.

A polymer that makes a strong bond with the microneedles may be used as a material to form the back substrate. The material used to form the back substrate may be rigid or flexible depending on the application. Suitable flexible materials include, but are not limited to, paper, textile, polyether ether ketone (PEEK), polyethylene terephthalate (PET), polyethylene naphthalate (PEN), polytetrafluoroethylene (PTFE), parylene, and polyimide. Elastic and flexible resins may also be used (e.g., Elastic 50A Resin (Part Number: FLELCL01), Flexible 80A Resin (Part Number: FLFL8001)). UV curable resins may also be used, such as when there is a need for conformality, flexibility, and elasticity in the microneedle patch. Hard resins may be used for applications having a need for rigid back substrates. A suitable example of a hard resin includes, but is not limited to, Surgical Guide Resin (Part Number: FLSGAM01).

In some embodiments, if the back substrate is planar or substantially planar, the “planar area” of the patch can be calculated as the area of the patch in the plane defined by the back substrate. In some such embodiments, the “microneedle planar area” of the patch can be calculated as the area of a regular polygon, an irregular polygon, a circle, or another suitable shape, wherein the area is defined as the largest area circumscribed by the locus of all lines: (1) in the plane of the back substrate and (2) that connect all microneedles in pairs. Stated more plainly, but without wishing to modify the foregoing geometric definition, the microneedle planar area is the area defined by the perimeter of the microneedles on the patch. In some embodiments, the planar area of the patch is about 0.50, 1.0, 2.0, 3.0, 4.0, 5.0, 6.0, 7.0, 8.0, 9.0, 10, 12, 15, 16, 18, 20, 24, 25, 27, 28, 30, 32, 33, 35, 36, 40, 42, 45, 48, 50, 55, 56, 60, 63, 64, 65, 70, 72, 75, 80, 81, 85, 90, 95, 99, 100, 105, 110, 120, 121, 125, 130, 135, 140, 144, 145, 150, 160, 170, 180, 190, 200, 210, 215, 220, or 225 cm². In some embodiments, the planar microneedles area of the patch is about 0.10, 0.20, 0.25, 0.30, 0.40, 0.50, 1.0, 2.0, 3.0, 4.0, 5.0, 6.0, 7.0, 8.0, 9.0, 10, 12, 15, 16, 18, 20, 24, 25, 27, 28, 30, 32, 33, 35, 36, 40, 42, 45, 48, 50, 55, 56, 60, 63, 64, 65, 70, 72, 75, 80, 81, 85, 90, 95, 99, 100, 105, 110, 120, 121, 125, 130, 135, 140, 144, 145, 150, 160, 170, 180, 190, 200, 210, 215, 220, or 225 cm². In some embodiments, the planar area of the patch is between about 0.10 and about 1.0, or between about 1.0 and about 5.0, or between about 1.0 and about 10, or between about 5.0 and about 10, or between about 10 and about 20, or between about 10 and about 100, or between about 20 and about 50, or between about 50 and about 100, or between about 100 and about 150, or between about 150 and about 200, or between about or 200 and about 250 cm². In some embodiments, the planar microneedle area of the patch is between about 0.10 and about 1.0, or between about 0.50 and about 100, or between about 1.0 and about 5.0, or between about 1.0 and about 10, or between about 5.0 and about 10, or between about 10 and about 20, or between about 10 and about 100, or between about 20 and about 50, or between about 50 and about 100, or between about 100 and about 150, or between about 150 and about 200, or between about 200 and about 250 cm².

### Use and Performance of Microneedles

In some examples, the present disclosure provides a system for sampling a fluid, e.g., ISF, of a subject using a patch including at least one microneedle. Specifically, at least one microneedle is coupled to a first side of a patch, and a sensor is coupled to a second side of the patch. The sensor is provided to detect a presence of at least one biomarker in a fluid that is drawn through the at least one microneedle. For example, a collection reservoir is also coupled to the patch, and the sensor is coupled to, e.g., disposed within, the collection reservoir. The collection reservoir is in fluid communication with the at least one microneedle, which includes at least one fluid channel therein to withdraw a fluid from a subject. In operation, a suction force is provided to the patch, i.e., the at least one microneedle, which in turn drives or directs fluid from a region of interest of a subject, through the fluid channel defined by the microneedle, and into the collection reservoir. The withdrawn fluid is then provided to the sensor, which may utilize a variety of analysis techniques to determine the presence and/or concentration of one or more biomarkers in the fluid. In this way, the sampling systems described herein provide a non-invasive, user-friendly, and compact option for sampling a fluid in a subject, which in turn can increase subject comfort and satisfaction.

More specifically, an interstitial fluid sampling system includes at least one grooved microneedle coupled to a first side of a back substrate or patch, and a reservoir is coupled to a second side of the patch such that the reservoir and the plurality of grooved microneedles are in fluid communication with one another. In some aspects, the system further includes a sensor provided as one or more of an absorbent bead or a removable cartridge, e.g., a paper-based sensor. The sensor is disposed within the reservoir and/or coupled to the plurality of grooved microneedles, In some aspects, the sensor is configured to retain a fluid received from the plurality of grooved microneedle. For example, an absorbent bead includes a one way valve that allows air to exit when compressed such that expansion of the absorbent bead to its original state creates suction through the plurality of grooved microneedles as the bead expands and the one way valve closes. Thus, the absorbent bead performs two functions, namely, providing a suction force to withdraw fluid and serving as a sensor to detect the presence and/or concentration of biomarkers in the fluid.

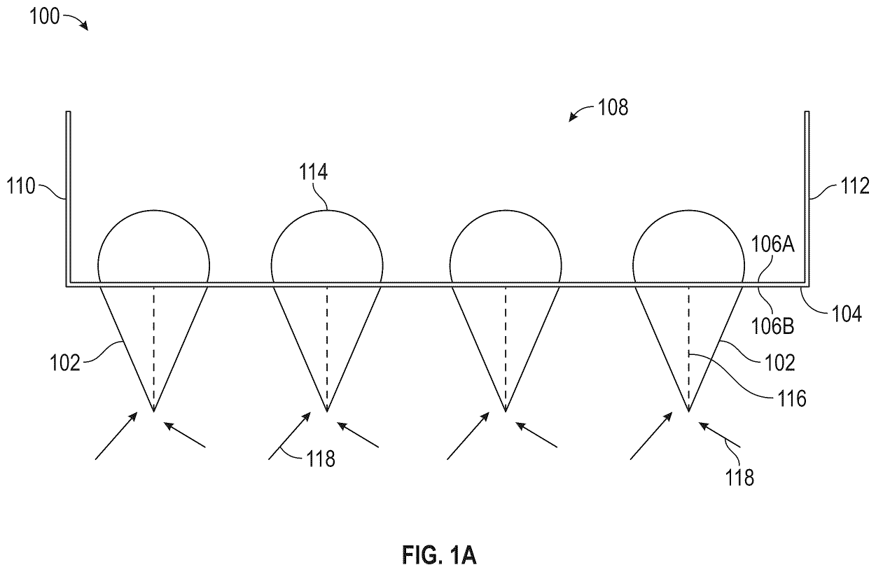

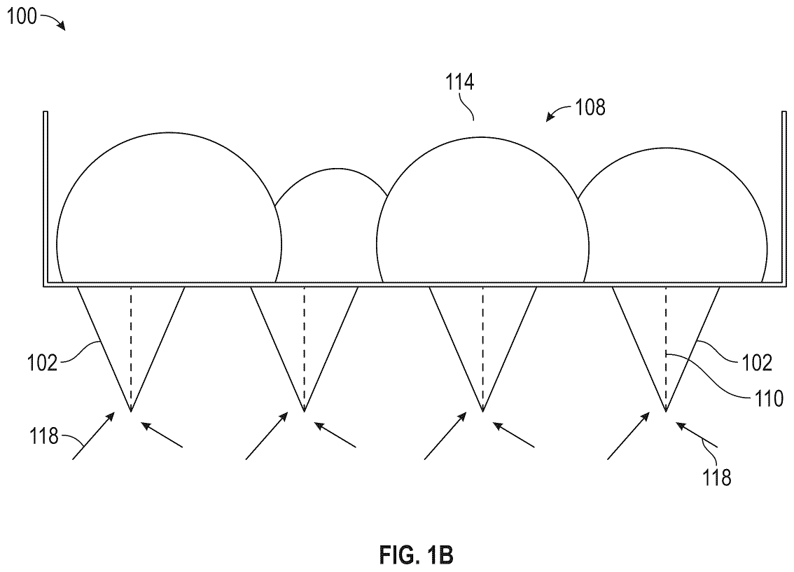

Referring now to FIG. 1A, an example sampling system, e.g., an interstitial fluid (ISF) sampling system 100, includes a grooved microneedle array 102 and a back substrate or patch 104, and the patch 104 has a first side 106A that defines a bottom wall of a collection reservoir 108. The collection reservoir 108 includes sidewalls 110, 112 to further define the collection reservoir 108. The grooved microneedle array 102 is coupled to a second side 106B of the patch 104, e.g., a side of the patch 104 that is opposite to the first side. In other examples, the collection reservoir 108 is coupled to another side of the patch 104, e.g., a lateral side. Additionally, absorbent beads 114 are connected to the first side 106A of the patch 104 such that the absorbent beads 114 are further in fluid communication with the grooved microneedle array 102. The absorbent beads initially exist in a non-expanded state. In some aspects, a single absorbent bead 114 is connected to a single grooved microneedle, or a single absorbent bead 114 is connected to multiple grooved microneedles. Connecting the absorbent beads 114 with the grooved microneedle array 102 causes a pressure differential to exist between the collection reservoir 108 and the grooved microneedle array 102 such that a suction force is applied through the grooves 116 of the grooved microneedles. The suction force encourages fluid motion in a direction represented by arrows 118 from the tips of the grooved microneedle array 102 into the absorbent beads 114.

In some aspects, a hydrophilic material is used for the absorbent beads 114. Some non-limiting examples of types of beads that can be used are agarose gels, PLGA, PCL, alginate gels, gelatin methacrylate gels, pHEMA, PNIPAAM, PDMS, hydrogels, ECOFLEX, rubber resins, elastomers, or polyacrylate beads. Additionally, it should be understood to one skilled in the art that the shape of the absorbent beads is not limited to the hemispherical examples shown in FIG. 1A, and that a variety of possible shapes of the absorbent beads 114 may exist to provide suction for sampling ISF.

Referring now to FIG. 1B, the direction of fluid motion represented by arrows 118, as encouraged by the suction force created by the absorbent beads 114, urges ISF to flow through the grooved microneedle array 102 up into the absorbent beads 114. In some aspects, the absorbent beads 114 are configured to retain the fluid and expand radially outward to reach an expanded conformation. Once the absorbent beads 114 have reached a fully expanded state, the absorbent beads 114 are recovered from the collection reservoir 108 and may further undergo downstream analysis that includes but is not limited to analysis regarding metabolic function, hormones, cytokines, chemokines, or genomic material. In some aspects, downstream methods of analysis of the beads includes extraction of ISF using centrifugation or another method of crushing the beads, using analytical instruments such as LC/MS, UPLC, ELISA, genomic sequencing, or other various instruments that would be readily known be one skilled in the art. As another non-limiting example, electrochemical techniques are used for analysis. In some aspects, sensing beads are used such as pH sensitive, glucose sensitive, lactate sensitive, and/or interleukin-6 (IL-6) beads which are functionalized with fluorescent or colorimetric dyes that react to an analyte of interest and provide useful information regarding the focuses of the analysis methods discussed above. In some aspects, the absorbent beads remain in the collection reservoir. In some aspects, the use of absorbent beads 114 is optional and ISF is be drawn into the collection reservoir using only capillary action of the grooved microneedle array 102.

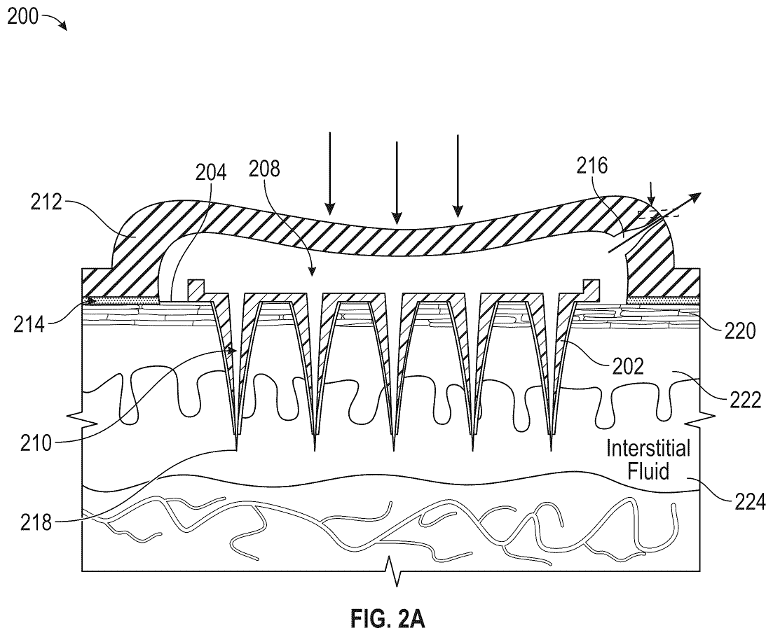

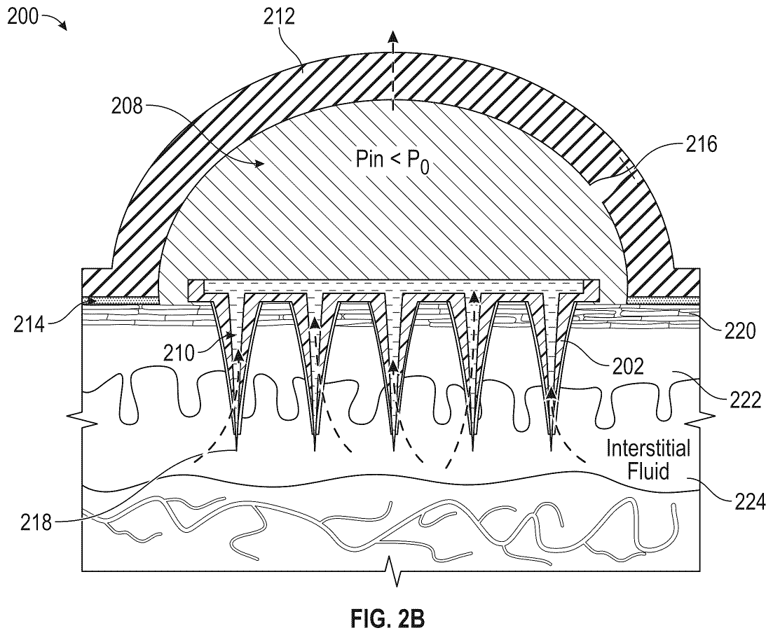

Referring now to FIG. 2A, another example sampling system 200 is illustrated which is similar to the system 100 illustrated in FIGS. 1A and 1B. For example, the sampling system 200 includes a grooved microneedle array 202, a patch 204, a collection reservoir 208, and/or grooves 210 defined by the microneedle array 202. In some aspects, the collection reservoir 208 is defined by an elastic membrane 212 which is secured to a skin sample of a subject via an adherent bottom layer 214. The membrane 212 also includes a one-way valve 216, e.g., a valve disposed within a side of the membrane 212, which defines an air channel between the collection reservoir 208 and an ambient environment such that air is able to exit the collection reservoir 208 when the membrane 212 is compressed. As discussed above, the membrane 212 is elastic, meaning that the membrane 212 is configured to return to its original, non-compressed shape after being compressed. As the membrane 212 decompresses, a suction force created by the expanding area of the collection reservoir 208 is applied along the grooves 210 of the microneedle array 202 to draw a fluid, e.g., ISF, up through the grooved microneedle array 202 and into the collection reservoir 208. In some aspects, the grooved microneedle array 202 is placed onto a skin sample of a subject such that tips 218 of microneedles of the array 202 extend through the stratum carenum 220, epidermis 222, and dermis 224 layers of the skin. In some aspects, the tips 218 of the array 202 are disposed within the dermis 224 layer of the skin. In some aspects, the membrane 212 is provided as part of an absorbent bead, e.g., the absorbent beads 114 discussed above for FIG. 1.

Referring now to FIG. 2B, the membrane 212 expands radially outward as ISF travels upward through the grooves 210 of the grooved microneedle array 202 via capillary action aided by the suction force created by the expanding membrane 212, thus driving the sampled ISF into the collection reservoir 208. Further, as the membrane 212 expands outward, the one-way valve 216 is closed, thereby closing the air channel with the ambient environment and allowing the membrane 212 to remain in a fully expanded state.

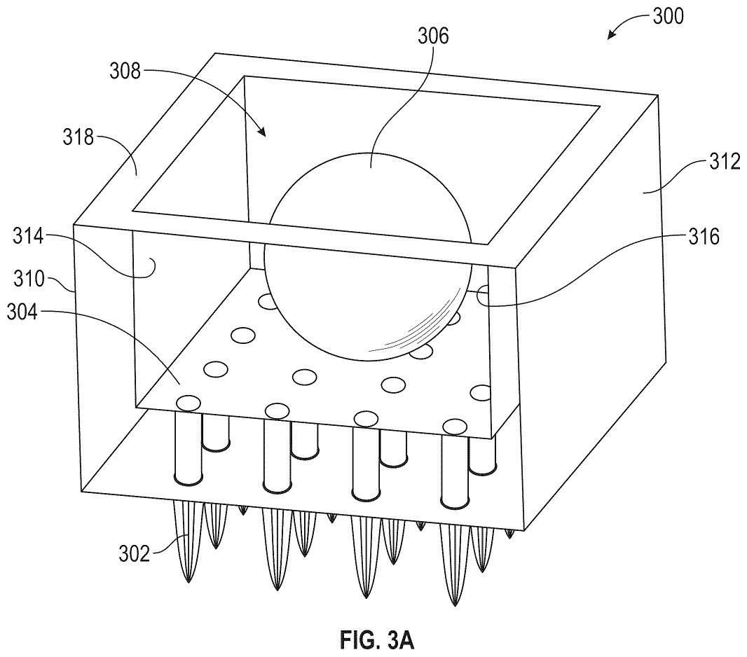

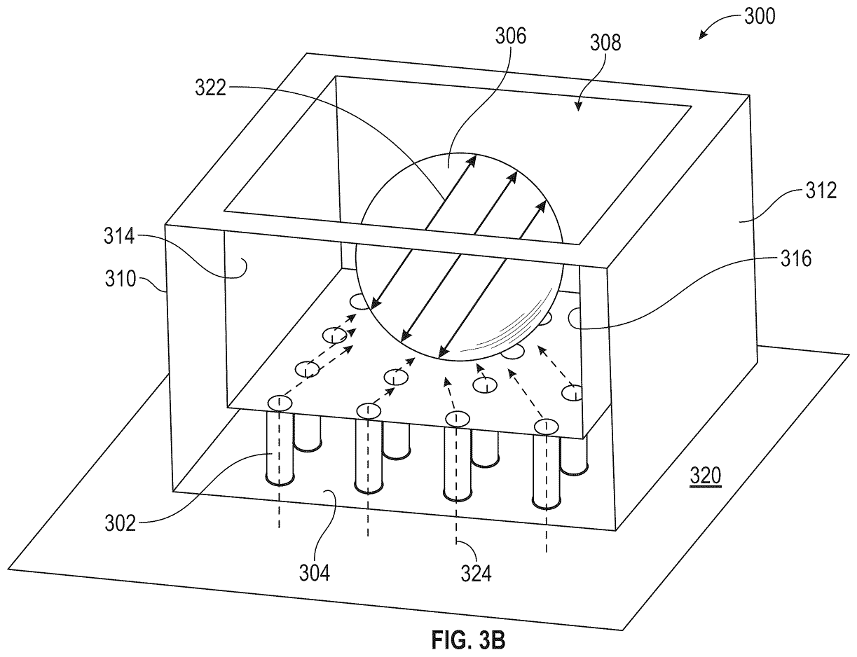

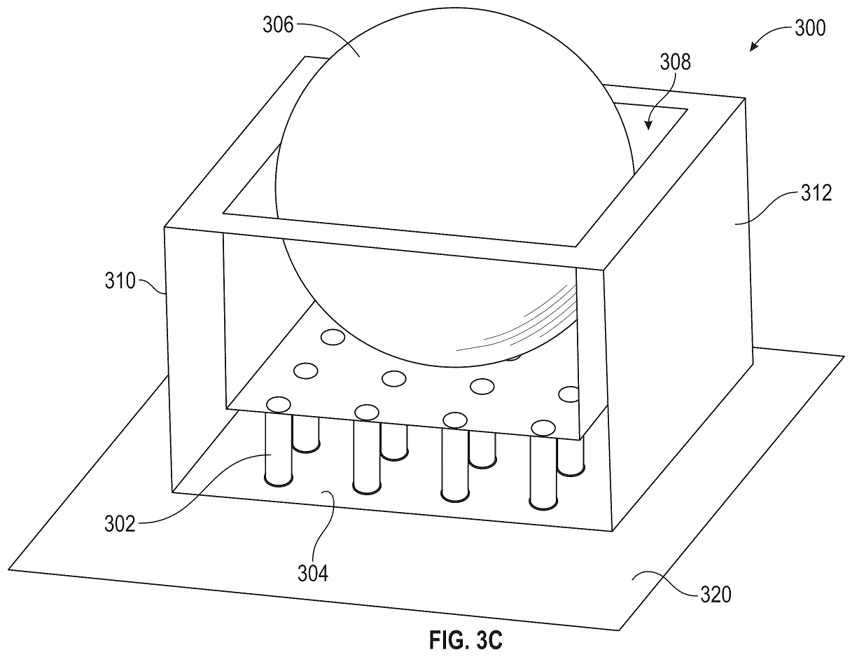

Referring now to FIG. 3A, a perspective view is illustrated of an example microneedle array 300. The grooved microneedle array 300 is configured to extract a fluid from an area of interest of a subject via a plurality of microneedles 302, and the microneedle array 300 is coupled to a patch 304. In some examples, the grooved microneedle array 300 is also coupled to an absorbent bead 306 which initially exists in a non-expanded state in a collection reservoir 308, e.g., a collection reservoir similar to those discussed above. In some aspects, the collection reservoir 308 is defined by exterior side walls 310, 312 and the patch 304 that is coupled to the grooved microneedle array 300. The exterior side walls 310, 312 are molded to interior side walls 314, 316 to define a rim 318 of the patch 304. In some aspects, the rim 318 has a width extending between exterior side wall 310 and interior side wall 314 of between 0.50 mm and 5.0 mm, or between 1.0 mm and 4.0 mm, or between 2.0 mm and 3.0 mm, or between 0.25 mm and 1.0 mm, or between 0.50 mm and 0.75 mm. In some aspects, the collection reservoir 308 is coupled to more than one absorbent bead 306 and extends along a length of the patch 304. In some aspects, the collection reservoir 308 comprises a biocompatible and photocurable hard resin, e.g., a polymer such as chitosan, chitosan polybutylene adipate terephthalate, or the collection reservoir 308 comprises a mold that extends from the patch 304 to define a unitary patch construction. However, it will be understood that the absorbent bead 306 may be optional in some examples, meaning that the grooved microneedle array 300 may be in direct fluid communication with the collection reservoir 308.

Referring now to FIGS. 3B and 3C, the grooved microneedle array 300 extends into a skin model 320 and utilizes capillary action to draw ISF in an upward direction represented by arrows 322 through grooves of the grooved microneedle array 300 which are connected to the absorbent bead 306. In some aspects, the absorbent bead 306 expands in a radially outward direction represented by arrows 322 as fluid is drawn through the grooves of the microneedles 302 and absorbs ISF. As illustrated in FIG. 3B, the absorbent bead 306 expands as it fills, with the ISF drawn in a direction represented by arrows 324 from the grooved microneedle array 300, towards the collection reservoir 308, and into the absorbent bead 306. In some aspects, the ISF travels through grooves of the grooved microneedle array 300. As illustrated in FIG. 3C, the suction force discontinues once the absorbent bead 306 reaches a fully expanded state, i.e., a filled state, although ISF may continue to travel upward through the grooved microneedle array 300 and into the collection reservoir 308 via capillary action of the grooves of the grooved microneedle array 300. As previously described, the absorbent bead 306 is removed from the collection reservoir 308 after reaching a fully expanded state and undergoes downstream analysis, or the absorbent bead 306 remains in the collection reservoir 308 and is a sensing bead that requires no additional instrumentation to assess properties of the sampled ISF. In some aspects, the collection reservoir 308 is itself removable from the patch 304 and/or the grooved microneedle array 300, meaning that the collection reservoir 308 may be removed from the patch 304 to undergo downstream analysis.

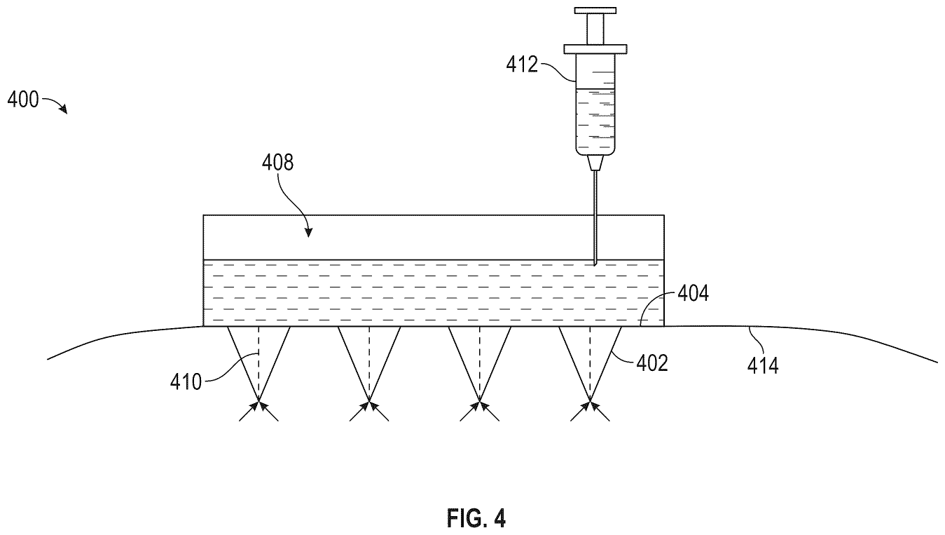

Referring now to FIG. 4, yet another example of a sampling system, e.g., an ISF sampling system 400, is illustrated. The system 400 includes at least a microneedle array 402, a patch 404, a collection reservoir 408, grooves 410 defined by the microneedle array 402, and a syringe pump 412. In some aspects, the syringe pump 412 is used to apply negative pressure, i.e., active pressure, to the collection reservoir 408. The negative pressure causes a suction force to be applied along grooves 410 of a grooved microneedle array 402 which in turn drives or directs ISF upward from skin 414 of a subject and through the grooves 410 of the grooved microneedle array 402 extending into the skin 414. In this way, ISF is sampled by the microneedle array 402 and deposited or retained within the collection reservoir 408. In some aspects, the syringe pump 412 is in direct contact with the collection reservoir 408, or the syringe pump 412 is indirectly connected to the collection reservoir 408. In some aspects, the syringe pump 412 is a laboratory syringe pump, a medical infusion pump, a siphon pump, a hand pump, or another type of pump, as discussed below.

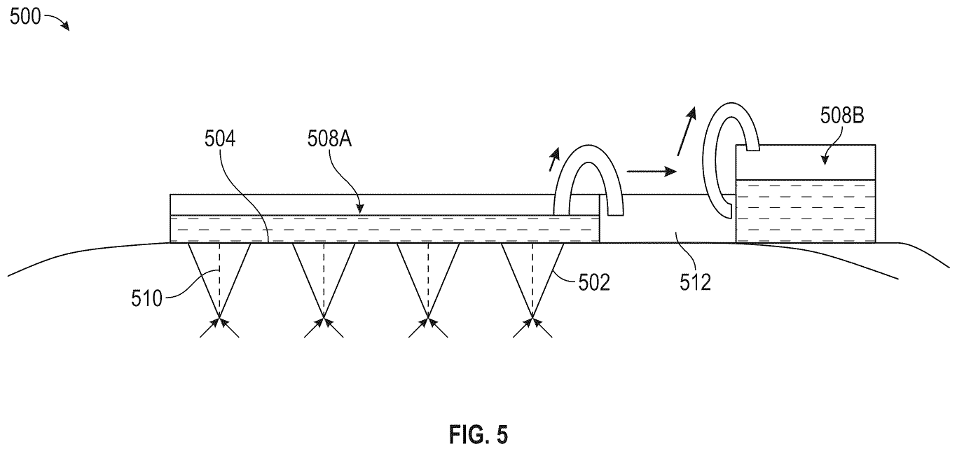

Referring now to FIG. 5, still another example of a sampling system e.g., an ISF sampling system 500, is illustrated. The system 400 includes at least a microneedle array 502, a patch 504, a first collection reservoir 508A, grooves 510 defined by the microneedle array 502, and a micropump 512. The micropump 512 is used to actively pump ISF upward and through the grooves 510 of the grooved microneedle array 502. As discussed above, ISF is drawn into the first collection reservoir 508A, e.g., via the micropump 512 and/or via capillary action of the grooves 510. In some aspects, the system 500 further includes a second collection reservoir 508B which is coupled directly to the first collection reservoir 508A, e.g., abutting a wall of the first collection reservoir 508A, or the second collection reservoir 508B is indirectly coupled to the first collection reservoir 508A. In some embodiments, the second collection reservoir 508B contains sensing agents to sample certain aspects of the ISF, such as those previously discussed.

In some aspects, the micropump 512 is an electrohydrodynamic pump, an electroosmotic pump, a peristaltic pump, a piezoelectric pump, a vacuum pump, a handheld pump (see Examples below), or another type of pump. In some aspects, the micropump 512 includes an electronic chip that may be configured to wirelessly transmit data, e.g., stored data and/or data acquired from a sensor connected to the first or second collection reservoirs 508, to an external device. For example, the electronic chip includes a radiofrequency identification (RFID) tag for identifying and/or tracing the biomarkers of interest in an ISF sample. In some other instances, a rate of ISF intake of the first and second collection reservoirs 508 is intermittently checked using the external device. The external device may be a smart phone, a wearable electronic device, an RFID tag reader, a laptop, or any other suitable wireless device.

In some aspects, the grooved microneedle array 502 comprises a thermosensitive material that exhibits a temperature dependent ISF uptake profile. For example, the grooved microneedle array 502 comprises a thermo-responsive material, such as poly(N-isopropylacrylamide) (PNIPAAM). In these instances, the electronic chip may be in communication with a heating/cooling source, e.g., a Peltier thermoelectric heating or cooling patch, a resistive conductive heater, or the like, and the electronic chip may be configured to modulate the temperature of the grooved microneedle array 502 using the heating/cooling source to increase or decrease the ISF uptake in a region of interest of a subject.

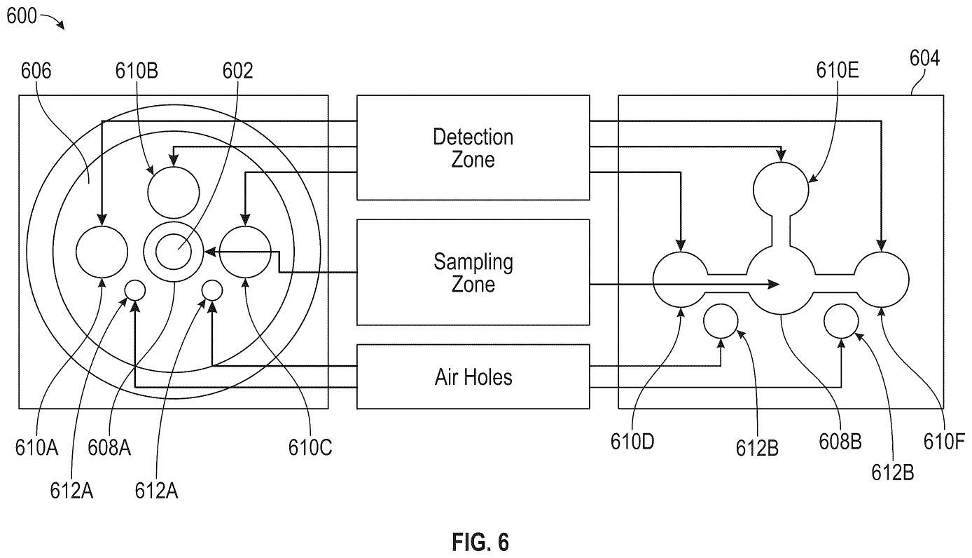

Referring now to FIG. 6, an example detection system 600 is illustrated including a microneedle array 602 and a sensor 604. As discussed above, a microneedle sampling system includes a sensor to analyze a fluid withdrawn from a subject. For example, a sensor is used to detect a presence of one or more biomarkers and/or a concentration of one or more biomarkers in a fluid sample that is withdrawn from a subject. It is contemplated that a sensor may be implemented in a variety of different ways, such as an absorbent sensing bead, as discussed above. In other examples, the sensor is implemented as a removable cartridge, e.g., a paper-based sensor.

Specifically, and with reference to the non-limiting example illustrated in FIG. 6, the sensor 604 is provided as a removable cartridge that is coupled to a rear side 606 of the microneedle array 602. It is contemplated that a collection reservoir (not shown) is also coupled to the rear side 606 of the microneedle array 602, such that the sensor 604 is disposed within the reservoir (not shown). Further, a first sampling zone 608A is disposed on the rear side 606 of the microneedle array 602, the first sampling zone 608A defining an area in which fluid withdrawn from the microneedle(s) (not shown) is deposited. In some aspects, the first sampling zone 608A is disposed in a center of the rear side 606, although it is contemplated that the first sampling zone 608A may be arranged in a variety of other locations in other examples. Further, one or more detection zones 610 are also disposed on the rear side 606 of the microneedle array 602, e.g., a first detection zone 610A, a second detection zone 610B, and a third detection zone 610C. Moreover, one or more apertures 612A extend through the rear side 606 of the microneedle array 602. In some aspects, the apertures 612A serve as air vents for the sensor 604, as will be discussed below.

With continued reference to FIG. 6, the sensor 604 is illustrated that is configured to be removably placed on the rear side 606 of the microneedle array 602. In some examples, the sensor 604 is a microfluidic device, e.g., a paper-based sensor, to perform molecular analysis of a fluid sampled by the microneedle array 602. To that end, the sensor 604 includes zones that are similar to those of the rear side 606 of the microneedle array 602. For example, the sensor 604 includes a second sampling zone 608B and one or more detection zones 610, e.g., a fourth detection zone 610D, a fifth detection zone 610E, and a sixth detection zone 610F. In some aspects, the sensor 604 also includes one or more apertures 612B that correspond to the apertures 612A of the microneedle array 602, e.g., apertures that serve as air vents.

During operation, the sensor 604 is placed on the rear side 606 of the microneedle array 602 such that the corresponding zones of the microneedle array 602 and the sensor 604 are aligned with one another. For example, the sensor 604 is arranged on the rear side 606 such that the apertures 612B of the sensor 604 are aligned with the apertures 612A of the microneedle array 602. Correspondingly, the first sampling zone 608A is placed in contact with the second sampling zone 608B, and the first, second and third detection zones 610A, 610B, 610C are placed in contact with the fourth, fifth, and sixth detection zones 610D, 610E, 610F, respectively. Thus, as fluid is withdrawn through the microneedles (not shown), the fluid is deposited in the first sampling zone 608A and is at least partially absorbed into the second sampling zone 608B of the sensor 604 via cohesion and/or absorption properties of the sensor 604. The fluid can then be driven, e.g., via capillary action, from the second sampling zone 608B to each of the surrounding detection zones 610 of the sensor 604. In some aspects, one or more enzymes are disposed within the detection zones 610, such that the fluid interacts with the enzyme(s) once the fluid reaches the detection zones 610.

Further, the resulting reaction provides an indication to an operator of a presence and/or concentration of a biomarker in the fluid. For example, the resulting reaction may alter a color of each detection zone 610 that can be used to determine presence and/or concentration of a biomarker. In some aspects, each detection zone 610 of the sensor 604 is configured to detect a different property or marker. For example, each detection zone 610 includes a colorimetric probe that is specific to each biomarker being measured. In this way, the detection system 600 provides a streamlined method of biomarker detection.

From the above, it will be understood that an ISF sampling system can be implemented in a variety of different ways and with a variety of different components to optimize sampling efficiency and accuracy. However, in general, the ISF sampling systems herein are provided to withdraw a fluid from a region of interest of a subject and analyze the withdrawn fluid to determine the presence and/or concentration of biomarkers therein. As discussed above, an ISF sampling system generally includes at least one microneedle which is inserted into a subject's skin, and ISF is withdrawn through a fluid channel defined in the microneedle. In some aspects, the fluid channel is provided as a groove that extends along a longitudinal axis of the microneedle, a groove disposed in an exterior surface of a body of a microneedle, a central channel that extends along a longitudinal axis of the microneedle, or any combination thereof.

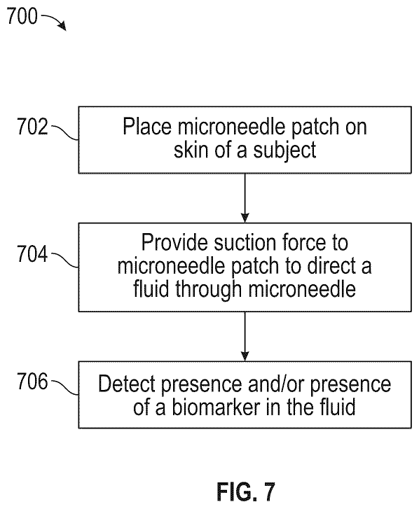

FIG. 7 illustrates a method of sampling ISF from a subject by driving ISF through one or more microneedles of a microneedle array, and analyzing the sampled ISF to detect biomarker presence and/or concentration therein. As discussed above, using a microneedle patch to sample a fluid from a subject provides a non-invasive, user-friendly, and compact option compared to traditional sampling techniques, e.g., hypodermic needles. Moreover, using microneedles for fluid sampling can also increase sampling duration and efficacy, as inserting microneedles into a subject's skin is relatively painless in comparison to traditional techniques, which in turn improves subject safety and satisfaction. In some aspects, a method of sampling interstitial fluid includes placing a microneedle patch on the skin of a subject, e.g., on a region of interest of the subject's skin, providing a suction force to the microneedle patch and driving a fluid through the microneedle array, and placing the fluid in contact with a sensor to detect biomarker presence and/or concentration.

For example, a process 700 of sampling ISF with a microneedle array or patch can include placing the microneedle patch on a subject's skin, e.g., a region of interest of a subject's skin, at step 702. As discussed above, a microneedle patch includes at least one microneedle coupled to a first side of the patch and, in some examples, a collection reservoir coupled to a second side of the patch. In some aspects, the microneedle includes at least one fluid channel therein, e.g., a central channel and/or an exterior groove, and the fluid channel is in fluid communication with the collection reservoir. At step 704, the process 700 includes providing a suction force to the microneedle patch to drive or direct a fluid through the at least one microneedle and into the collection reservoir. Put another way, a suction force urges fluid through the fluid channel of the microneedle toward the patch and the collection reservoir. In some aspects, the suction force is provided by a pump, e.g., a vacuum pump, a syringe pump, a micropump, etc., that is coupled to the collection reservoir. In some examples, the suction force is provided by compressing a flexible membrane to impart negative pressure on the collection reservoir. Further, it is contemplated that the suction force is aided by capillary action of the fluid channel of the microneedle, such that the fluid is also passively driven toward the collection reservoir, as discussed above.

In addition, the process 700 includes detecting a presence and/or a concentration of a biomarker in the fluid that is withdrawn from the subject via the microneedle patch at step 706. In some aspects, biomarker detection is provided by a sensor that is coupled to the collection reservoir, or biomarker detection is performed after the microneedle patch and/or collection reservoir is removed from the subject's skin for downstream analysis. In some examples, the sensor is an absorbent bead, i.e., a sensing bead, that is configured to retain the fluid therein, or the sensor is a removable cartridge that is disposed within the collection reservoir. For example, withdrawn or sampled fluid that is contained within the collection reservoir is conducted to a paper-based cartridge sensor via capillary action, e.g., a sample zone on the paper-based sensor. In some aspects, the paper-based sensor includes a detection zone that is configured to change in color to indicate presence and/or concentration of a biomarker, or the paper-based sensor includes multiple such detection zones that each correspond to a different biomarker. Thus, in some aspects, detecting a presence and/or concentration of a biomarker includes analyzing a color of the detection zone(s) of the sensor. Accordingly, a sampling system including a microneedle patch can be used to non-invasively and efficiently sample a fluid, e.g., ISF, of a subject. Moreover, the sampling system disclosed herein streamlines analysis of a withdrawn fluid, obviating the need for complex equipment and improving clinical applicability. That is, the system offers an easy-to-use option for ISF sampling that also enhances subject safety and satisfaction.

### Microneedle Structure

Herein disclosed, inter alia, is a class of microneedles called grooved or hollow microneedles. Grooved microneedles are hard microneedles with a groove that can facilitate fluid motion along the groove via capillary action. At the same time, the grooved microneedles disclosed herein possess excellent structural properties. The fabricated microneedles are resistant to breakage, having a high Young's modulus (expected 1000 times higher than human skin) and can effectively penetrate a variety of skin types without breaking. The microneedles can form and maintain an exceptional pointed tip (see Examples below).

In some aspects, the microneedle includes a conically shaped body defined by an exterior side and extending between a base and an apex point. The base has a center point, and a center line of the microneedle defines a longitudinal axis that intersects the center point of the base and the pointed apex. Further, the base defines an outer diameter of the microneedle. In some examples, a groove or recess is defined in the conically shaped body. The groove is an axial groove that extends substantially parallel with respect to the longitudinal axis of the microneedle, or the groove is offset from the longitudinal axis. In other examples, the groove is not a linear groove, such as a spiral groove that wraps around the body of the microneedle. In some aspects, the groove including a first or right groove face and a second or left groove face. In some aspects, the first or right groove face and the second or left groove face extend between the exterior side of the conically shaped body and the center line. The first or right groove face includes a first or right intersection point with the base, and the second or left groove face includes a second or left intersection point with the circular base. In some aspects, the microneedle includes more than one groove, e.g., two, three, four, five, or more than five grooves.

As discussed above, the microneedle defines a fluid channel therein, and the fluid channel can be configured to draw ISF therethrough. In some aspects, the microneedle includes multiple fluid channels, e.g., channels that are in fluid communication with one another. For example the microneedle includes a central channel that extends at least partially along the longitudinal axis. The central channel may also be in fluid communication with apertures defined in the base and/or body of the microneedle. For example, the central channel is in fluid communication with the groove defined in the body, e.g., a groove disposed on the exterior side of the body, such that a fluid pathway comprising the central channel and the groove is formed within the microneedle. In some aspects, the central channel defines a channel length, the groove defines a groove length, and both the channel length and the groove length are measured in a direction that is parallel with respect to the longitudinal axis.

In some embodiments, the outer diameter, i.e., the diameter of the base, of the microneedle is between about 0.20 mm and about 5.0 mm, or between about 0.20 mm and about 4.0 mm, or between about 0.20 mm and about 3.0 mm, or between about 0.20 mm and about 2.0 mm, or between about 0.20 mm and about 1.0 mm, or between about 0.20 mm and about 0.75 mm, or between about 0.20 mm and about 0.50 mm, or between about 0.40 mm and about 0.50 mm, or between about 0.50 mm and about 1.0 mm, or between about 0.80 mm and about 1.2 mm, or between about 1.0 mm and about 2.0 mm, or between about 1.0 mm and about 3.0 mm. In some embodiments, a maximum grooved aperture length (i.e., a length that extends between the first or right intersection point and the second or left intersection point) is between about 0.10 mm and about 3.0 mm, or between about 0.10 mm and about 2.0 mm, or between about 0.10 mm and about 1.0 mm, or between about 0.10 mm and about 0.75, or between about 0.10 mm and about 0.50 mm, or between about 0.10 mm and about 0.30 mm, or between about 0.20 mm and about 0.30 mm, or between about 0.20 mm and about 0.50 mm, or between about 0.25 mm and about 0.75 mm, or between about 0.25 mm and about 1.0 mm, or between about 0.50 mm and about 0.75 mm, or between about 0.75 mm and about 1.0 mm, or between about 0.50 mm and about 1.0 mm, or between about 0.50 mm and about 1.25 mm, or between about 0.75 mm and about 1.5 mm.

In some embodiments, a maximum axial length of the microneedle (i.e., a length that extends between the center point of the base and the apex point, measured in a direction that is parallel with respect to the center line of the microneedle) is between about 0.50 mm and about 10 mm, or between about 0.50 mm and about 8.0 mm, or between about 0.50 mm and about 6.0 mm, or between about 0.50 mm and about 5.0 mm, or between about 0.50 mm and about 4.0 mm, or between about 0.50 mm and about 3.0 mm, or between about 0.50 mm and about 2.0 mm, or between about 0.50 mm and about 1.5 mm, or between about 0.50 mm and about 1.0 mm, or between about 1.0 mm and about 1.5 mm, or between about 0.75 mm and about 1.0 mm, or between about 1.0 mm and about 1.25 mm, or between about 0.80 mm and about 1.2 mm, or between about 0.90 mm and about 1.1 mm, or between about 0.10 mm and about 1.0 mm, or between about 0.10 mm and about 0.75 mm, or between about 0.10 mm and about 0.50 mm.

In some embodiments, the channel length of the microneedle is between about 10% and about 100% of the maximum axial length, or between about 50% and about 100% of the maximum axial length, or between about 75% and about 100% of the maximum axial length, or between about 85% and about 95% of the maximum axial length. In some embodiments, the groove length of the microneedle is between about 10% and about 100% of the maximum axial length, or between about 25% and about 75% of the maximum axial length, or between about 40% and about 60% of the maximum axial length, or about 50% of the maximum axial length.

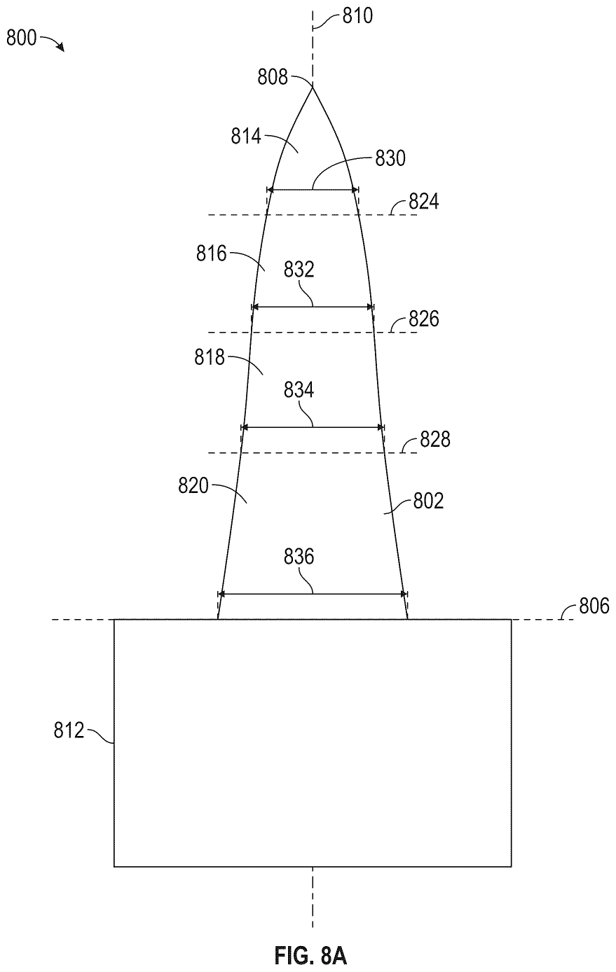

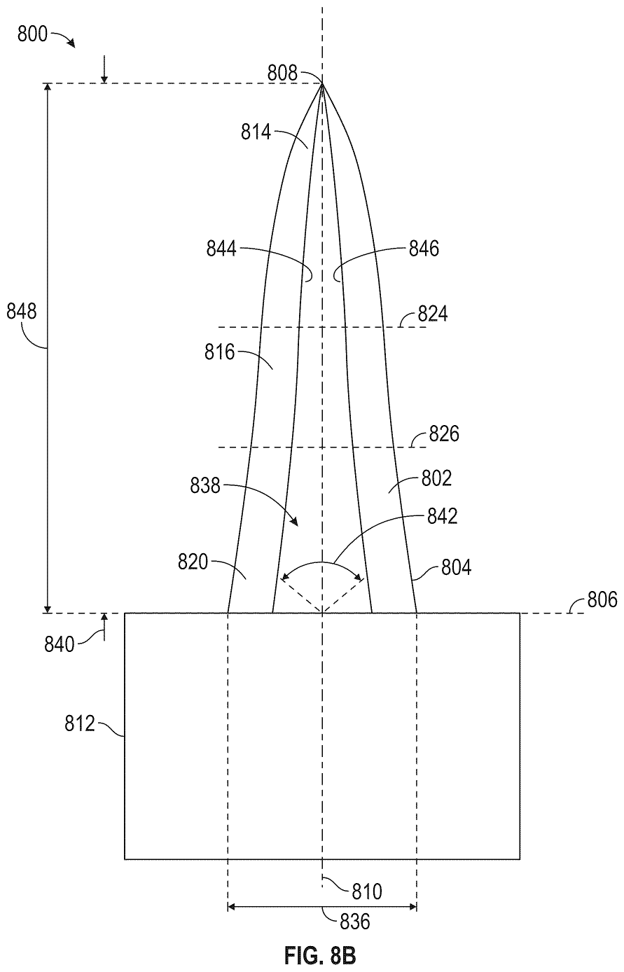

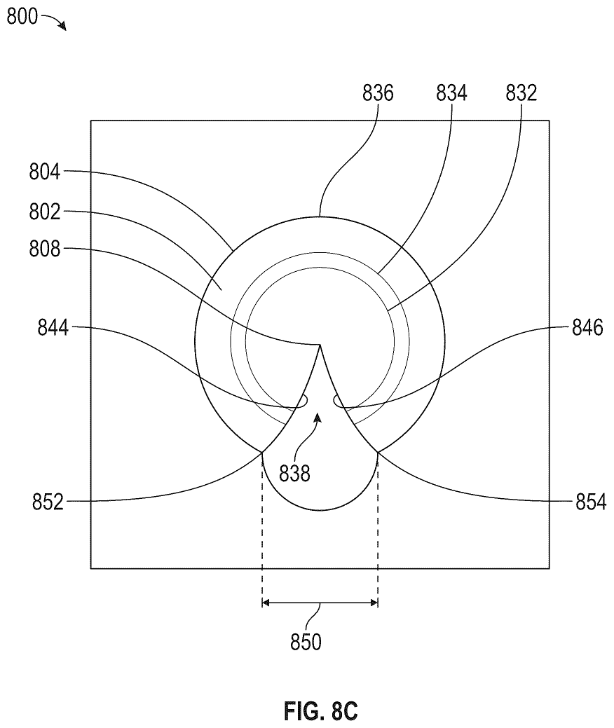

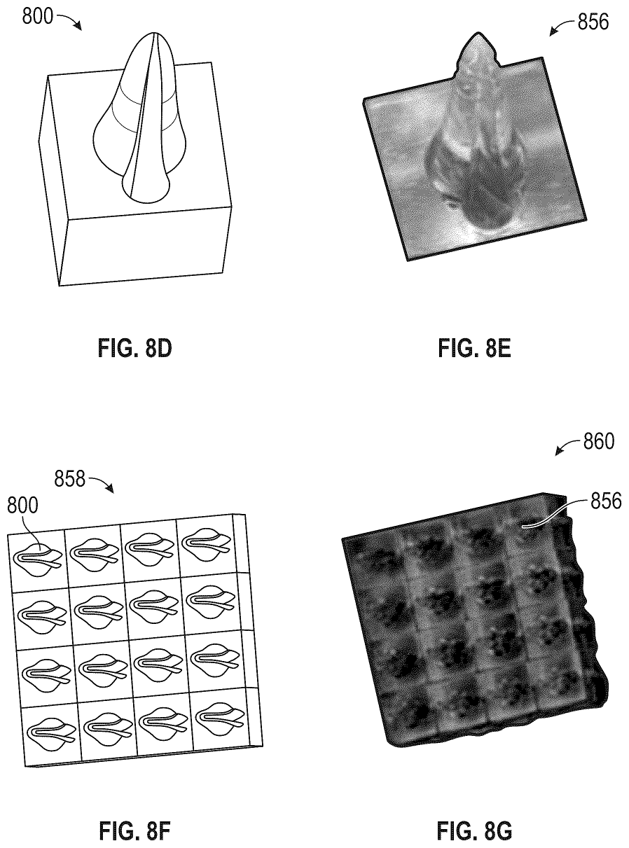

Referring now to FIG. 8A, a rear view of an example grooved microneedle 800 is illustrated. In some embodiments, the grooved microneedle 800 has a body 802 that includes an exterior surface 804. The grooved microneedle 800 extends from a base plane 806 to an apex point 808, and the body 802 is substantially conical in shape. A longitudinal axis 810 of the grooved microneedle 800 extends through the grooved microneedle 800 at the apex point 808 in a direction that is perpendicular with respect to the base plane 806. In some aspects, the grooved microneedle 800 comprises different sections including a tip portion 814, a first intermediate portion 816, a second intermediate portion 818, and/or a base portion 820, or the grooved microneedle 800 includes only the tip portion 814, the first intermediate portion 816, and the base portion 820. In some aspects, the grooved microneedle 800 includes only one section, i.e., a base portion or a tip portion. The exterior surface 804 extends along the exterior of each section of the grooved microneedle 800. It should be readily understood to one skilled in the art that any combination of possible sections can be used to form the grooved microneedle 800 and that the grooved microneedle may include sections other than those discussed herein.

In some aspects, the exterior surface 804 is substantially conical in shape at the tip portion 814, which extends from the apex point 808 to a first transition plane 824. The first transition plane 824 is parallel with respect to the base plane 806 and defines an interface between the tip portion 814 and the first intermediate portion 816. Further, the tip portion 814 has a tip base diameter 830 at the first transition plane 824. In some aspects, the tip base diameter 830 is in a range of between 0.05 and 0.50 mm, or between 0.10 and 0.40 mm, or between 0.15 and 0.3 mm, or between 0.20 and 0.25 mm. The exterior surface 804 is substantially convexly curved or sigmoidal in shape at the first intermediate portion 816, which extends from the first transition plane 824 to a second transition plane 826. The second transition plane 826 is parallel with respect to the base plane 806 and defines an interface between the first intermediate portion 816 and the second intermediate portion 818, or between the first intermediate portion 816 and the base portion 820. The first intermediate portion 816 has an outer diameter 832 at the second transition plane 826 that is in a range of between 0.10 and 0.6 mm, or between 0.15 and 0.50 mm, or between 0.20 and 0.40 mm, or between 0.25 and 0.35 mm, or between 0.25 and 0.30 mm. In some aspects, the exterior surface 804 is substantially convexly curved, sigmoidal, or concavely curved in shape at the second intermediate portion 818, which extends from the second transition plane 826 to a third transition plane 828. The third transition plane 828 is parallel with respect to the base plane 806 and defines an interface between the second intermediate portion 818 and the base portion 820. The second intermediate portion 818 has an outer diameter 834 at the third transition plane 828 that is in a range of between 0.10 and 0.6 mm, or between 0.15 and 0.50 mm, or between 0.20 and 0.40 mm, or between 0.25 and 0.35 mm, or between 0.30 and 0.35 mm. In some aspects, the exterior surface 804 is substantially convexly curved, sigmoidal, or concavely curved in shape at the base portion 820, which extends from the third transition plane 828 to the base plane 806. Further, the base plane defines an interface between the base portion 820 and a patch or back substrate 812, and the base portion 820 has an outer diameter 836 at the base plane 806 that defines the base diameter of the microneedle, as discussed above. In some aspects, the first intermediate portion 816 interfaces directly with the base portion 820, meaning that the second intermediate portion 818 may be optional.

In some examples, the tip base diameter 830 is between about 25% and about 75% of the outer diameter 836, or between about 40% and about 60% of the outer diameter 836, or about 50% of the outer diameter 836. In some examples, the outer diameter 832 of the first intermediate portion 816 is between about 25% and about 75% of the outer diameter 836 of the base portion 820, or between about 50% and about 75% of the outer diameter 836 of the base portion 820, or about 60% of the outer diameter 836 of the base portion 820. In some examples, the outer diameter 834 of the second intermediate portion 818 is between about 50% and about 100% of the outer diameter 836 of the base portion 820, or between about 60% and about 80% of the outer diameter 836 of the base portion 820, or about 75% of the outer diameter 836 of the base portion 820.

Referring now to FIG. 8B, a front view is illustrated of the example grooved microneedle 800. As shown, the body 802 of the grooved microneedle 800 includes the tip portion 814, first intermediate portion 816, and base portion 820. As discussed above, a microneedle can include a groove or recess therein which can define at least a portion of a fluid channel within the microneedle. In the illustrated non-limiting example, a groove 838 is disposed within the body 802 of the grooved microneedle 800, and the groove 838 is disposed along at least a portion of a maximum axial length 840, i.e., a length that is measured from the base plane 806 to the apex point 808 along the longitudinal axis 810. The groove 838 is formed in the body 802 and defines a fluid channel between the apex point 808 and base plane 806. In some aspects, the groove 838 defines a groove angle 842 that is measured along a radial line (not shown) that extends perpendicularly outward with respect to the longitudinal axis 810. In particular, the groove angle 842 is measured between a first groove wall 844 and a second wall 846 defined by the body 802, the groove angle 842 represented by curved double arrow 842. In some aspects, the groove 838 extends through only the base portion 820 or only the base portion 820 and the first intermediate portion 816 or the base portion 820, first intermediate portion 816, and tip portion 814. In some aspects, the body 802 includes more than one groove and/or a central channel that is communication with the groove 838, as will be discussed below in greater detail.

In some examples, the groove 838 defines a groove length 848 that is measured in a direction that is parallel with respect to the longitudinal axis 810, and the groove length 848 is between about 1% and about 100% of the maximum axial length 840, or between about 25% and about 100% of the maximum axial length 840, or between about 50% and about 100% of the maximum axial length 840, or between about 75% and about 100% of the maximum axial length 840, or at least about 50% of the maximum axial length 840, or at least about 75% of the maximum axial length 840. Referring now to FIGS. 8A and 8B, the outer diameter 836 of the base portion 820 is between about 25% and about 75% of the maximum axial length 840, or between about 25% and about 50% of the maximum axial length 840, or about 40% of the maximum axial length 840, in some examples. Further, the outer diameter 836 of the base portion 820 is between about 25% and about 75% of the maximum axial length 840, or between about 40% and about 60% of the maximum axial length 840, or about 50% of the maximum axial length 840, in some examples.

FIG. 8C illustrates a top view of the example grooved microneedle 800 including a maximum groove aperture length 850 defined as a length extending between a first point 852 on the first groove wall 844 and a second point 854 on the second wall 846, as represented by arrows 850. In some aspects, the maximum groove aperture length 850 includes length ranges as discussed above. In the illustrated non-limiting example, the groove 838 extends from the exterior surface 804 of the body 802 between the first groove wall 844 and the second wall 846 and to the longitudinal axis 810 (see FIG. 8A). While the first groove wall 844 and second wall 846 are substantially curved in an inward direction toward the groove channel, one skilled in the art would readily understand that the groove walls 844, 846 may include other configurations, i.e., outwardly curved, straight, angled, and/or other configurations, to define the groove 838. Correspondingly, it is contemplated that the groove 838 may not extend entirely to the longitudinal axis 810 (see FIG. 8A), meaning that the groove 838 may be provided as a surface groove, in some examples.

Referring now to FIG. 8D, a perspective view of the single grooved microneedle 800 is illustrated. Using processes similar to those described herein, the single grooved microneedle 800 is used as a design for manufacturing a molded microneedle 856 as illustrated in FIG. 8E. In some aspects, the molded microneedle 856 is formed using 3D Nanoscribing. In some aspects, the single grooved microneedle 800 is part of a microneedle array 858 as illustrated in FIG. 8F, which is used as a design for manufacturing a molded microneedle array 860 as illustrated in FIG. 8G. While the microneedle arrays 858, 860 of FIGS. 8F and 8G, respectively, are illustrated as square arrays, e.g., 4×4 arrays, it will be understood that microneedles can be arranged in any suitable configuration to define a microneedle array. For example, a microneedle array can be provided as a square array, e.g., a 4×4 grid, a rectangular array, e.g., a 3×4 grid, a circular array, or another suitable configuration, as discussed below.

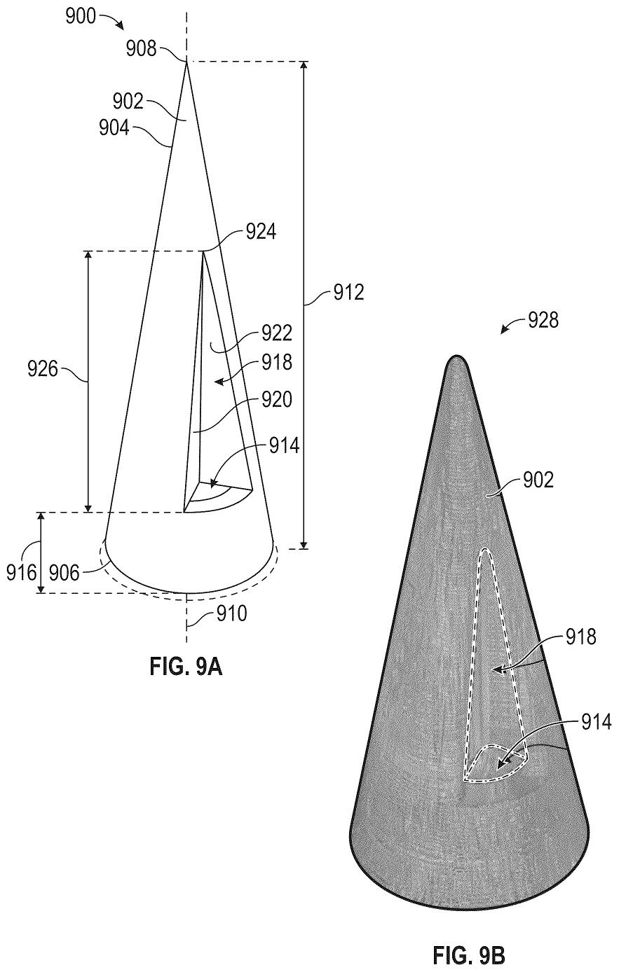

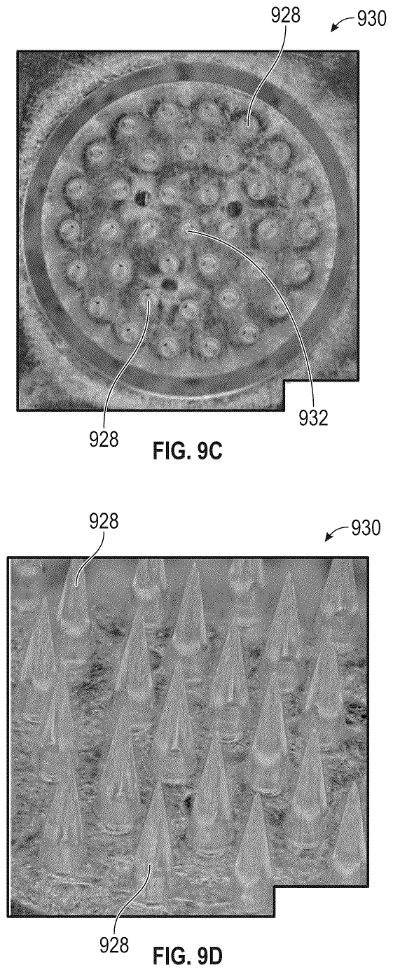

Referring now to FIGS. 9A and 9B, another example is illustrated of a microneedle 900 which includes multiple fluid channels therein. In some aspects, the microneedle 900 is similar to the microneedle 800 illustrated in FIGS. 8A-8C. Referring specifically to FIG. 9A, an isometric view of the microneedle 900 is illustrated, the microneedle 900 having a body 902 that defines an exterior surface 904, a base 906, e.g., a first end, and an apex point 908, e.g., a second end that is opposite the first end. Further, a longitudinal axis 910 extends through the microneedle 900, e.g., through a centerline (not shown) of the microneedle 900 that extends through the base 906 and the apex point 908. The microneedle 900 defines a maximum axial length 912 that extends between the base 906 and the apex point 908 and is measured in a direction that is parallel with respect to the longitudinal axis 910. However, the microneedle 900 differs from the microneedle 800 illustrated in FIGS. 8A-8C in some aspects.

For example, and with continued reference to FIG. 9A, the body 902 of the microneedle 900 is substantially conical in shape, and a central channel 914 is defined within the body 902. Specifically, the central channel 914 is axially aligned with the longitudinal axis 910 and defines a channel length 916 therealong, that is, a length measured in a direction that is parallel with respect to the longitudinal axis 910. In some examples, the body 902 includes one or more apertures (not shown) that are defined in the base 906, and the central channel 914 is in fluid communication with the apertures (not shown). As discussed above, a base of a microneedle is generally coupled to a patch and/or collection reservoir, so including apertures in the base of the microneedle can allow the microneedle to be in fluid communication with the patch and/or collection reservoir. In the illustrated non-limiting example, one or more apertures can also be defined in the exterior surface 904 of the body 902 so as to provide a path for fluid to flow through the microneedle 900. For example, a groove 918 is defined within the body 902, and the groove 918 is in fluid communication with the central channel 914. That is, the groove 918 defines an opening or aperture in the body 902 of the microneedle 900 which can serve as a site of fluid intake when the microneedle 900 is inserted into a subject's skin to sample ISF. In this way, the central channel 914, the groove 918, and/or any apertures defined in the body 902 and/or base 906 comprise a fluid pathway through which a fluid of a subject can be driven for sampling purposes, e.g., using the pumping mechanisms discussed above.

In some aspects, the groove 918 is a longitudinal groove that is similar in shape to the grooves illustrated in FIGS. 8A-8C, or the groove 918 defines a different shape. In the illustrated non-limiting example of FIG. 9A, the groove 918 defines a substantially triangular or pyramidal shape. Specifically, the groove 918 is defined by a first groove wall 920 and a second groove wall 922, the groove walls 922, 920 defined by the body 902. In some aspects, the groove walls 922, 920 follow the profile of the exterior surface 904 such that the groove walls 922, 920 converge at a groove tip 924. In some aspects, the groove 918 defines a groove length 926 that extends between the central channel 914 and the groove tip 924, measured in a direction that is parallel with respect to the longitudinal axis 910. In some examples, the channel length 916 is between about 5% and about 100% of the maximum axial length 912, or between about 5% and about 25% of the maximum axial length 912, or between about 10% and about 20% of the maximum axial length 912, or about 15% of the maximum axial length 912. In some examples, the groove length 926 is between about 25% and about 100% of the maximum axial length 912, or between about 25% and about 75% of the maximum axial length 912, or about 50% of the maximum axial length 912, as discussed above.

Referring now to FIG. 9B, a perspective view illustrated of a molded microneedle 928 that is manufactured using the processes described below. For example, the molded microneedle 928 is formed using 3D Nanoscribing based on the design of the grooved microneedle 900 illustrated in FIG. 9A. As illustrated the molded microneedle 928 includes the central channel 914 and the groove 918 that are defined, in part, by the body 902. In some aspects, the molded microneedle 928 is part of a molded microneedle array 930, as illustrated in FIGS. 9C and 9D In particular, FIG. 9C illustrates a top view of the microneedle array 930, and FIG. 9D illustrates a detail view of a portion of the molded microneedle array 930. In some aspects, the microneedle array 930 is a circular array, and the molded microneedles 928 are arranged in concentric circles about a center point 932 of the microneedle array 930. For example, the microneedle array 930 can include one, two, three, four, five, or more than five concentric circles of molded microneedles 928. Four such concentric circles are illustrated in the non-limiting example of FIG. 9C. As discussed above, it will be understood that microneedles can be arranged in any suitable configuration to define a microneedle array. For example, a microneedle array can instead be provided as a square array, e.g., a 4×4 grid, a rectangular array, e.g., 3×4 grid, or another suitable configuration, as discussed herein.

### Manufacture of Microneedles

In some embodiments of the present disclosure, a method of fabricating a grooved microneedle array includes a molding process, such as an additive molding process in which one or more materials are sequentially added to a series of molds. In addition, the methods disclosed herein can include applying one or more surface treatments during the molding process to enhance the material characteristics of the molded products. For example, a method of fabricating a grooved microneedle array includes laser cutting one or more grooved microneedle-shaped depressions into a first material to provide a first mold, and casting a second material onto the first mold to fill the depressions with the second material. The method further includes include curing the second material to provide a second mold having one or more grooved microneedles formed therein, and removing the cured second mold from the first mold. The method further includes applying a surface treatment, e.g., a plasma surface treatment, to a surface of the cured second mold, applying a release layer to the surface of the cured second mold, casting a third material onto the surface of the second mold, curing the third material to provide the microneedle mold, and removing the microneedle mold from the cured second mold to provide a microneedle patch.

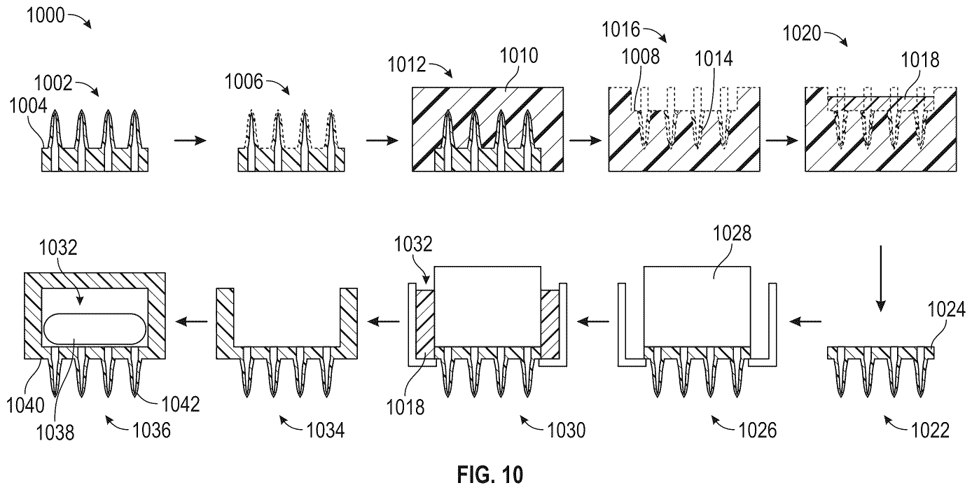

Referring now to FIG. 10, an example process 1000 for creating a flexible microneedle patch is illustrated. As shown in process block 1002, a microneedle master mold 1004 with a high groove aspect ratio is fabricated by using two-photon polymerization that utilizes an acrylic sheet such as an IP-S resin. The master mold is then provided with a surface treatment as shown in process block 1006. The microneedle master mold 1004 is then used to create a duplicate high-resolution microneedle mold 1008 using a moldable microneedle material 1010 as shown in process block 1012. In some aspects, the moldable microneedle material 1010 comprises a silicone elastomer such as a polydimethylsiloxane (PDMS) solution. In other instances, the moldable microneedle material 1010 comprises any other suitable moldable material for creating hard microneedles (i.e., microneedles having a Young's moduli significantly higher than that of human skin). After the moldable microneedle material 1010 is cast onto the microneedle mold 1008, excess moldable microneedle material 1010 is removed from the microneedle mold 1008, such that the moldable microneedle material 1010 fills only microneedle-forming cavities 1014 of the microneedle mold 1008 as shown in process block 1016.

After the excess moldable microneedle material 1010 is removed, with the microneedle-forming cavities 1014 being filled with the moldable microneedle material 1010, a biocompatible and photocurable hard resin 1018 is added and allowed to cure onto the microneedle mold 1008 after being placed in a vacuum chamber to remove bubbles from the liquid state PDMS, as shown in process block 1020. In process block 1022, the resulting PDMS mold 1024 is peeled from the microneedle mold 1008 to form grooved microneedle forming cavities and treated with oxygen plasma before being salinized. Salinization prevents the PDMS mold 1024 from sticking to the casting pre-polymer making it easily detachable. In process block 1026, backside reservoir walls 1028 are fabricated by placing an outer ring mold and a reservoir mold around the PDMS mold 1024 using ECOFLEX structures. In process block 1030, the biocompatible and photocurable hard resin 1018 is deposited in a reservoir cavity 1032 defined between the reservoir walls 1028. The PDMS mold 1024 with the ECOFLEX structures is then placed in a vacuum and is allowed to cure in process block 1034 before the ECOFLEX structures are removed. In process block 1036, an absorbent bead 1038 is inserted into the reservoir cavity 1032 which is then sealed using a biocompatible and photocurable hard resin molding method similar to that previously described. A resultant flexible microneedle patch 1040 comprising a final mold structure including the reservoir cavity 1032, grooved microneedles 1042, and the absorbent bead 1038, is then used for transdermal sampling of ISF.

The flexibility of the flexible microneedle patch 1040 allows the flexible microneedle patch 1040 to readily conform to any portion of the human body (e.g., arm, knee, neck, etc.), while the hardness of the grooved microneedles 1042 allows them to effectively penetrate the skin of a subject to permeate the skin for drug administration. Further, using the above-described method (shown in FIG. 10), flexible microneedle patches of varying sizing and including varying numbers of microneedles can be created. Accordingly, sampling and sizing can be adjusted accordingly for a given application.

### EXAMPLES

### Example 1

### Introduction

Interstitial fluid (ISF) provides a minimally invasive source of biomarkers for real-time health monitoring. Microneedles (MN) s, an array of high aspect ratio hollow micro-scale needles, are ideal for ISF sampling. Continuous ISF sampling often relies on micropumps to maintain negative pressure for suction, however, fabricating them is expensive and time consuming. Passive capillary action can also provide for sampling using narrow, closed or hollow microchannels. The following discussion describes a facile, high-resolution, and cleanroom-free technique to fabricate grooved microneedles for ISF sampling using capillary action and sustained by a sodium polyacrylate hydrogel bead. Prior approaches for fabrication of microneedles utilize traditional laser micromachining which is not suitable to fabricate hollow microneedles. Additionally, this method is expensive and time consuming. Grooved MN designs are easier to realize than hollow microneedles and have been reported earlier using lower-resolution 3D fabrication techniques. The lack of high aspect ratio grooves resolution limits the strength of capillary action needed for ISF sampling.

### Materials and Methods

To address the aforementioned shortcomings of reported lower-resolution 3D fabrication techniques, and as illustrated in FIG. 10, a master mold grooved MN array with high aspect ratio was fabricated by using two-photon polymerization using IP-S resin and an asymmetric crossover line pattern via laser cutting. This master mold was then used to create a duplicate high-resolution mold in Polydimethylsiloxane (PDMS), which was then cast using photocurable biocompatible hard resin to realize the final grooved MN array. A molding approach makes this scalable for large volume production. A sodium polyacrylate-based bead was placed on the grooved microchannels as an absorbent to sustain ISF sampling, through capillary action of the grooves. The ISF was then recovered from the swollen sodium polyacrylate beads.

A single grooved MN as illustrated in FIGS. 8A-8C was designed in SolidWorks® (Dassault Systems), with the following dimensions: height 1.2 mm, base diameter 0.47 mm, maximum grooved aperture 0.24 mm. An array of 4×4 MNs was then fabricated, as illustrated in FIGS. 8D-8G (Nanoscribe GmbH), with total width, length, and height MN array dimensions of 3.6 mm, 3.6 mm, and 1.95 mm, respectively. This master mold was replicated by casting PDMS on the master mold, put in a vacuum chamber to remove bubbles from the liquid-state PDMS, and cured for 3 hours in an oven at 60° C. The resulting PDMS mold was treated with oxygen plasma and then silanized with trichloro(1H, 1H,2H,2H-perfluorooctyl) silane. The silanization prevents the mold from sticking to the casting pre-polymer, making it easily detachable. Biocompatible/photocurable hard resin (Dental SG) from Formlabs (Somerville, MA, USA) was then cast on the PDMS MN mold, put in a vacuum chamber to remove the bubbles from the liquid-state resin, then thermal and UV cured in Form Cure (by Formlabs) at 60° C. for an hour, and finally detached from the mold.

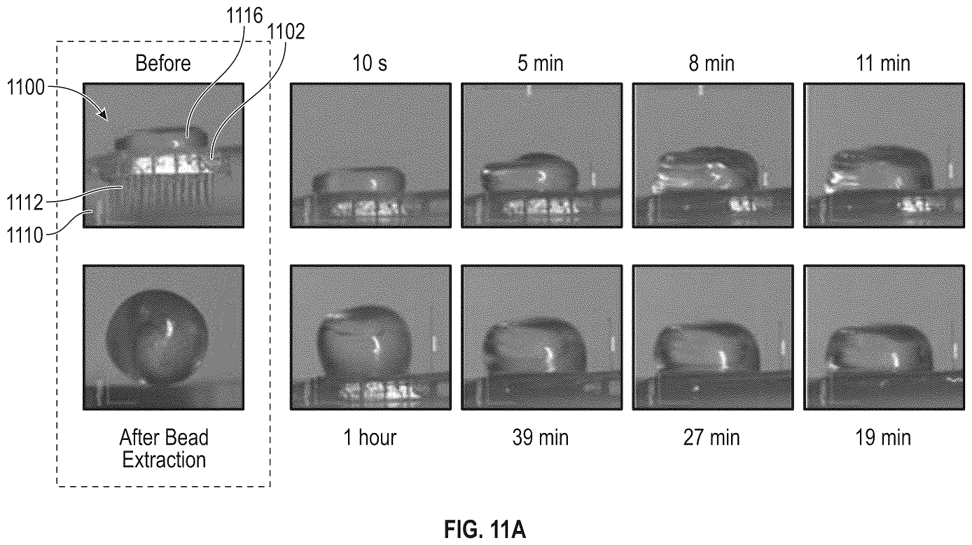

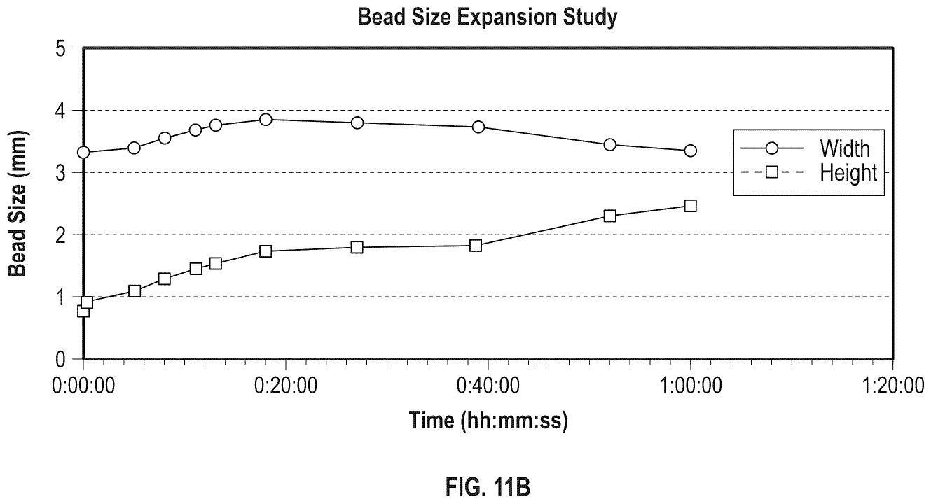

The backside reservoir walls to hold absorbent beads were then fabricated by placing two molds, one for the outer ring and the other for the reservoir using ECOFLEX structures. Bio-compatible/photo-curable resin (Dental SG) was deposited, vacuumed, and cured. Finally, a swellable sodium polyacrylate beads was inserted and the reservoir sealed using a biocompatible/photocurable hard resin sheet. The biocompatible resin is a polymer in a group comprising chitosan, chitosan polybutylene adipate terephthalate, poly(butylene adipate-co-terephthalate), polyethylene glycol, poly(ethylene glycol) diacrylate, gelatin, gelatin methacryloyl, polyvinyl alcohol, and silk. The assembled MN patch is ready for transdermal sampling. As shown in FIG. 11A, sampling kinetics of the MN patch 1100 were studied using a model skin gel 1110 made from agarose phantom gel. For easy visualization, the skin gel 1110 was loaded with Rhodamine B dye, which is sampled into the bead 1116 through grooved MNs 1112 attached to a back substrate 1102 of the MN patch 1100, validating the continuous sampling process. Data of the bead's size increment versus time was collected and analyzed as shown in FIG. 11B, proving the workability of the proposed grooved MNs.

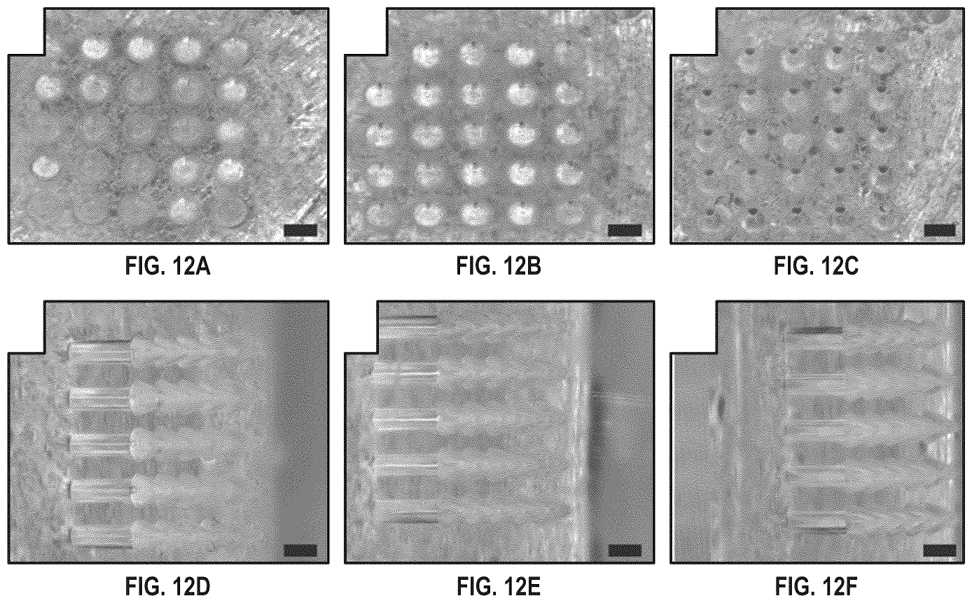

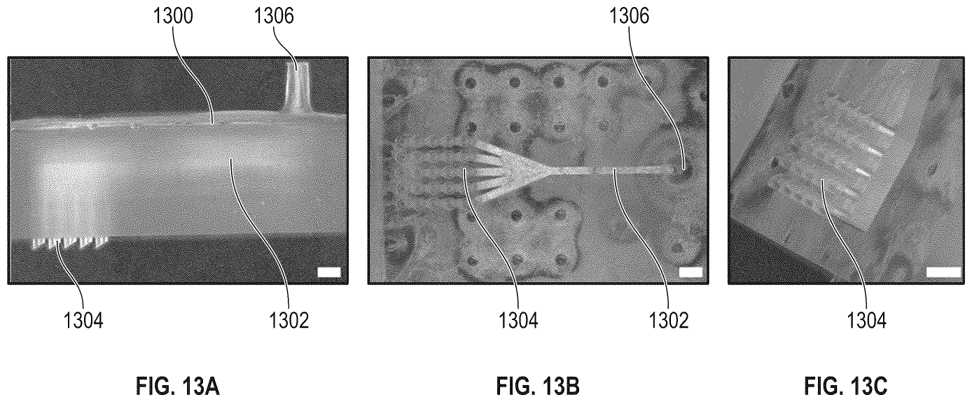

Using the methods of manufacturing grooved MNs as described above, an MN array with a 40 μm wide microfluidic channel was manufactured to be used to controllably sample and test ISF fluid for different biomarker concentrations. By connecting the MN through a microfluidic array, it is possible to distribute the small amounts of sampled ISF across multiple biomarker sensors (i.e., cortisol, IL-6, DHEA-S, D-serine, etc.). Three MN arrays with different groove dimensions of 50 μm, 100 μm, and 200 μm wide channels were also fabricated as shown, and top views of these MN arrays are illustrated in FIGS. 12A-12C, respectively. Side views of the 50 μm, 100 μm, and 200 μm wide channel MN arrays are illustrated in FIGS. 12D-12F, respectively. The connected 3D printed hollow MN array mold 1300 with the 40 μm wide microfluidic channel 1302 for ISF sampling are illustrated in FIGS. 13A-13C. The microfluidic channel 1302 is connected to a plurality of grooved microneedles 1304 at one end and an outlet 1306 at the other end for fluid recovery. The passive grooved MN arrays were designed using SolidWorks® and 3D printed using an Asiga Max X27 UV printer and PlasClear V2 resin (Asiga, Alexandria, Australia). In some aspects, COL lithography may also be used for MN fabrication.

To simulate the properties of skin ex vivo for a skin penetration test, a 10% gelatin eutectogel (made using a deep eutectic solvent system of choline chloride, ethylene glycol, and water mixed in a 1:2:1 molar ratio (Sigma Aldrich, Burlington, MA)) was used. The fluid sampling capabilities of the MNs was demonstrated using a 1 mg/ml solution of sulforhodamine B dye (Sigma Aldrich, Burlington, MA) in deionized water. For the passive sampling of fluid, the MN patch was dipped in the sulforhodamine B solution.

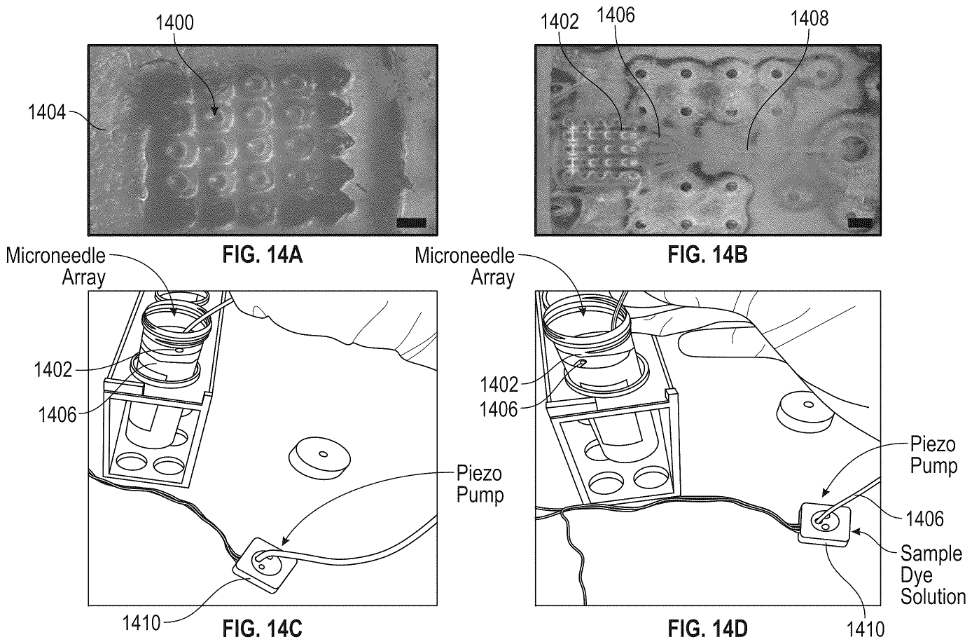

The amount of fluid naturally drawn into the microfluidic channel was then visually observed. For the active sampling of fluid, the 3D printed microneedle array was connected via flexible tubing to the inlet of a Takasago Piezoelectric Micro Pump (Model: SDMP302D). Then, the MN arrays were inserted into the sulforhodamine B solution and the voltage source connected to a micropump (Model: HY30005, Mastech) was turned on, initiating fluid flow through the MNs. The voltage applied to the micropump was 5V. The dye was successfully sampled at a rate of 3 ml/min, as illustrated in FIGS. 14A-14D. Specifically, a puncture pattern 1400 from inserting the MN arrays 1402 into the gelatin eutectogel 1404 representing a skin sample is illustrated in FIG. 14A which demonstrates that the stiffness and design of the 3D printed hollow microneedles appears to adequately pierce the gel 1404. After gel piercing and passively sampling the sulforhodamine B solution 1406, the MN array 1402 was shown to successfully draw the fluid 1406 into a microfluidic channel 1408, as illustrated in FIG. 11B. Further, FIGS. 11C and 11D illustrate the experimental setup before and after actively sampling the sulforhodamine B solution 1406 using a piezoelectric pump 1410, respectively.

### Example 2

### Materials and Methods

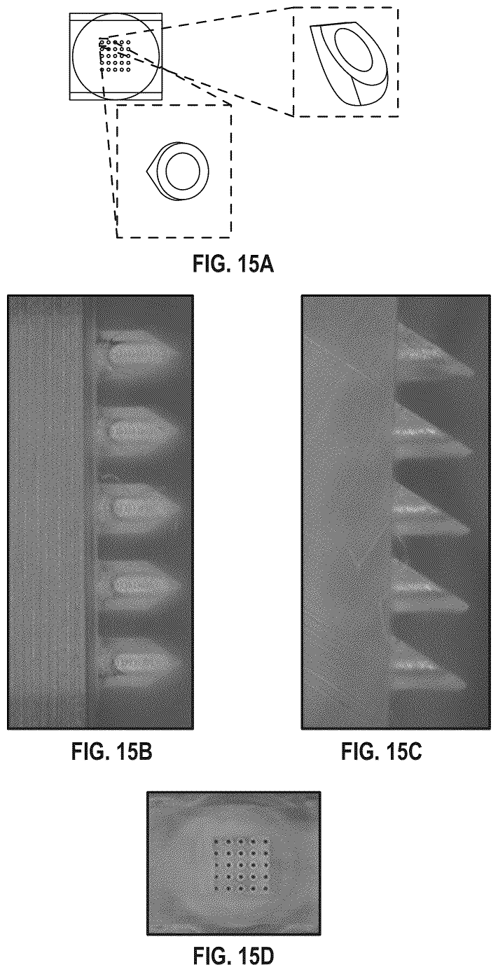

Sampling of ISF was demonstrated using a commercially procured vacuum suction device in conjunction with 3D printed hollow microneedles. Further, various design aspects of the microneedles have been optimized for active sampling of ISF. First, the microneedle patch tip angle was reduced from 60 degrees to 30 degrees to increase skin penetration performance, as illustrated in FIGS. 15A-15D. The microneedle patch's base channel length was also reduced from 2.0 mm to 1.0 mm, for the ISF to reach the reservoir faster. Referring specifically to FIGS. 15B and 15C, the microneedle includes an angled or slanted body rather than a conical body, such that the microneedle tip resembles the tip of a hypodermic needle. In some aspects, this angled body design enhances a sharpness of the microneedle array and leads to more efficient fluid uptake. Further, the height and width, e.g., 0.6 mm and 0.5 mm, respectively, of the microneedles were not modified. The microneedles and vacuum holder were designed using Solidworks, and 3D printed using an Asiga Max X27 UV printer and PlasClear V2 resin (Asiga, Alexandria, Australia).

Second, a vacuum holder was redesigned with a sliding platform on its tip for the hollow microneedles to be inserted. Additionally, the vacuum system path was modified compared to the imaging system. With this design, the sampled ISF will flow in the direction of the vacuum guided along the sides rather than in front of a camera using a suction pump. As a result, this design advantageously avoids fluid from spreading throughout an inner chamber, or collection reservoir, once sampling is complete. A nitrile O-ring with inner and outer diameter of 6.0 mm and 8.0 mm respectively, was inserted in between the microneedle patch and vacuum holder to seal the system.

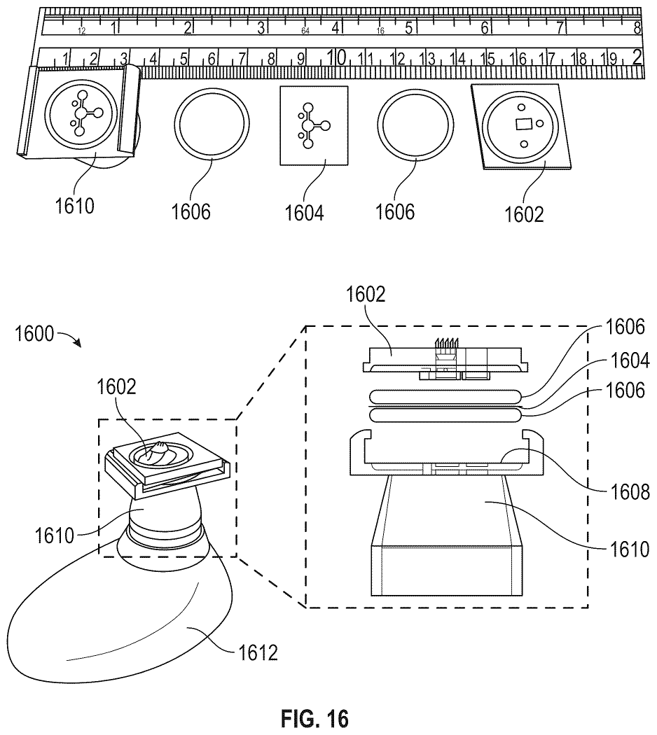

In an effort to perform real-time detection of biomarkers from the sampled ISF, the hollow microneedle system was integrated with a microfluidic paper-based (μPAD) sensing platform for the simultaneous colorimetric detection of different biomarkers in the ISF. Referring now to FIG. 16, a device 1600 included a 3D-printed hollow microneedle patch 1602 with a height of 1.5 mm, a base diameter of 0.6 mm, and a tip angle of 30 degrees to ensure skin penetration. A μPAD 1604 was aligned under the microneedle patch 1604 to collect an ISF sample and perform the sensing. The device 1600 was assembled as follows: first, the paper-based sensor 1604 was centered between two O-rings 1606, which were then aligned under the 3D-printed hollow microneedle patch 1602. The O-rings 1606 were then carefully slid into a top section 1608 of a vacuum holder 1610. The next step is to verify that an underside of the hollow MNs patch 1602 and the paper-based sensor 1604 are still aligned, e.g., by aligning zones with the patch 1602 with zones of the sensor 1604, as discussed above (see FIG. 16). The paper-based sensor 1604 was repositioned by carefully moving it from its edges. Finally, a commercially available mini-vacuum 1612 was connected to the bottom-side of the 3D-printed vacuum holder 1610. The two O-rings 1606 were placed in-between the paper-based sensor 1604 each of the patch 1602 and the vacuum holder 1610 to guarantee negative pressure inside the platform.

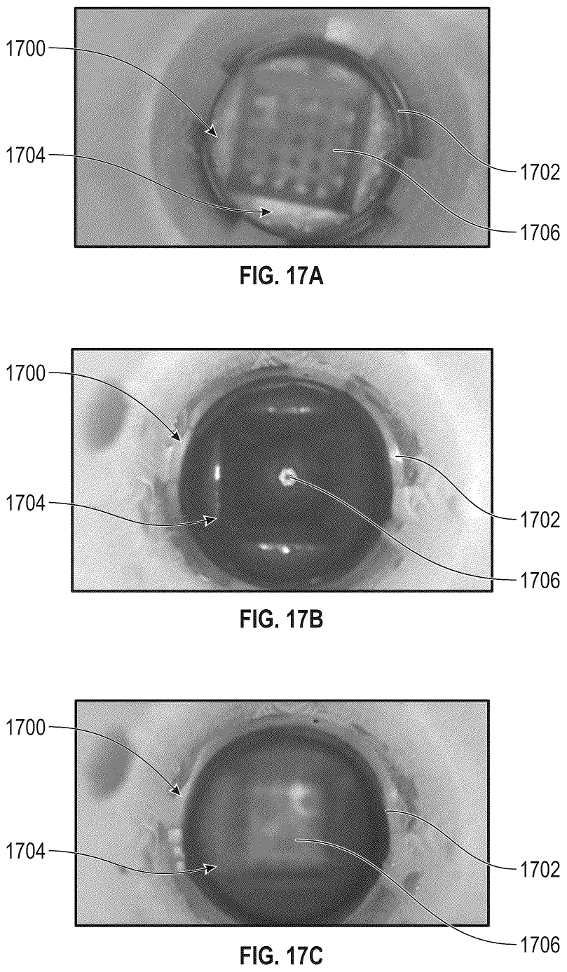

To demonstrate the utility of the device, fluid was sampled from commercially purchased chicken skin. The leg tissue of the chicken was first primed with a dye solution (Natural Red, Sigma Aldrich). The fluid was introduced right underneath the skin-muscle interface with a hypodermic needle. For the active sampling, the 3D printed microneedle patch was assembled to the vacuum holder, using the O-ring, and to the vacuum suction pump before being inserted into the skin while being stretched. The amount of fluid naturally drawn into the microfluidic channel was then visually observed using an internal camera of the vacuum pump at different time points, as illustrated in FIGS. 17A-17C. In particular, FIGS. 17A-17C illustrate an internal volume 1700 of a reservoir 1702 atop a sampling zone 1704 defined by a microneedle patch 1706. As illustrated in FIG. 17A, the internal volume 1700 is initially empty, i.e., filled with 0 μL of sampled solution, and in FIG. 17B, the vacuum pump is turned on to partially fill the reservoir 1702, i.e., with about 40 μL of sampled solution. The reservoir 1702 was completely full, i.e., filled with 50 μL of sampled solution, in FIG. 17C. The dye solution was successfully sampled at a rate of 30 μL/min. After stopping the suction pump and removing the microneedles from the skin, 20 μL of the sampled solution was retrieved with a micropipette. Further, 10 hollow microneedles and one vacuum suction device were used to sample ISF from mice.

### Results

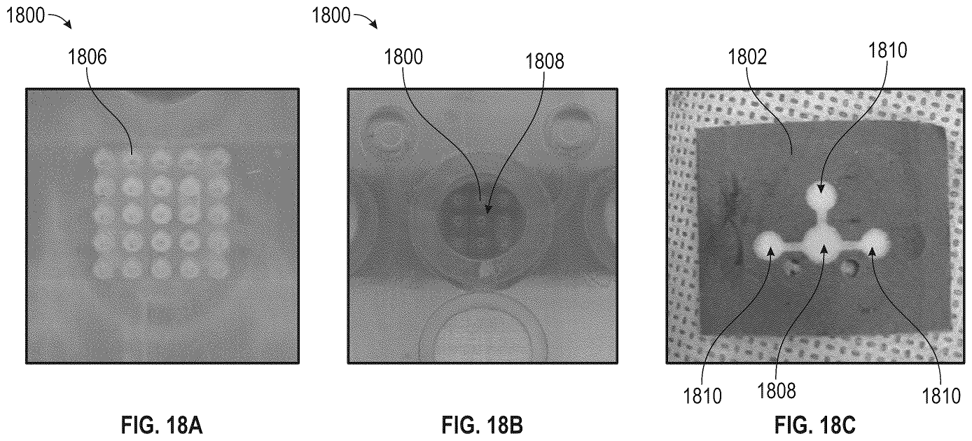

Referring now to FIGS. 18A-18C, the fluid sampling capabilities of the microneedle device were demonstrated using a 2% agarose gel prepared in 1×PBS. 0.1 mg of Sulforhodamine B was added to the agarose gel for visualization. A hollow microneedle patch 1800 containing a paper-based sensor 1802 was inserted into agarose gel (not shown) and a vacuum system (not shown) was turned on initiating fluid flow from the gel (not shown) through the microneedles 1806 into a sampling zone 1808 on the μPAD or sensor 1802. It was possible to draw the fluid into the sampling zone 1808 without directly touching a detection zone 1810 of the sensor 1802. The dye solution was drawn to the detection zones 1810 through capillary action, as illustrated in FIG. 18C. While these findings are encouraging, further in vitro and in vivo studies are necessary to validate the proposed platform.

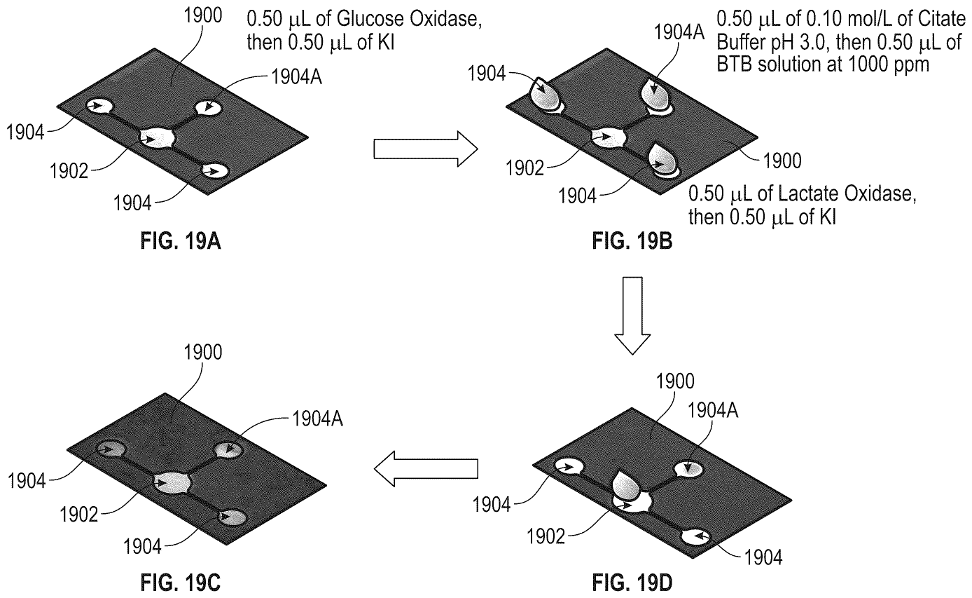

As a proof of concept, a simple μPAD sensor was designed for detecting glucose, lactate, and interleukin-6 (IL-6) to work in combination with a hollow microneedle system, as discussed above. The sensor was designed in Adobe Illustrator and printed with a wax printer (Xerox Color Cube 8580) to pattern a microfluidic channel. As illustrated in FIGS. 19A-19D, the prepared μPAD 1900 comprises one sample zone 1902 and three detection zones 1904. In this example, enzymatic reactions were selected to determine glucose and lactate due to their high selective analysis. Hydrogen peroxide (H₂O₂) was produced from the specific enzymatic reaction of these enzymes, and H₂O₂ subsequently reacted with pre-deposited reagents on the developed μPAD 1900. 0.50 μL of glucose oxidase (GOx) enzyme and lactate oxidase (LOx) enzyme was immobilized in each detection zone 1904 of glucose and lactate monitoring, and allowed to dry at room temperature (RT). Then, 0.50 μL of potassium iodide (KI) was added in the detection zones 1904 as a colorimetric probe, and allowed to dry at RT. For IL-6 monitoring, the dry-binding protein method was performed due to its cost-effectiveness and simple operating principle. In an assay, 0.50 μL of citrate buffer solution of pH 3.0 was coated onto a detection zone 1904A of IL-6 measurement. After drying at RT, 0.50 μL of bromothymol blue (BTB) solution was deposited into the same detection zone 1904A, and allowed to dry at RT, as indicated illustrated in FIG. 19B. The solution containing glucose, lactate, and IL-6 was introduced into the sample zone 1902, as illustrated in FIG. 19C. The paper in the detection zones 1904 turned from colorless yellowish-brown for the glucose and lactate, where the color intensities were proportional to their concentrations. In some aspects, the distance of coloration in the detection zone 1904A of IL-6 measurement was proportional to the concentration of IL-6.

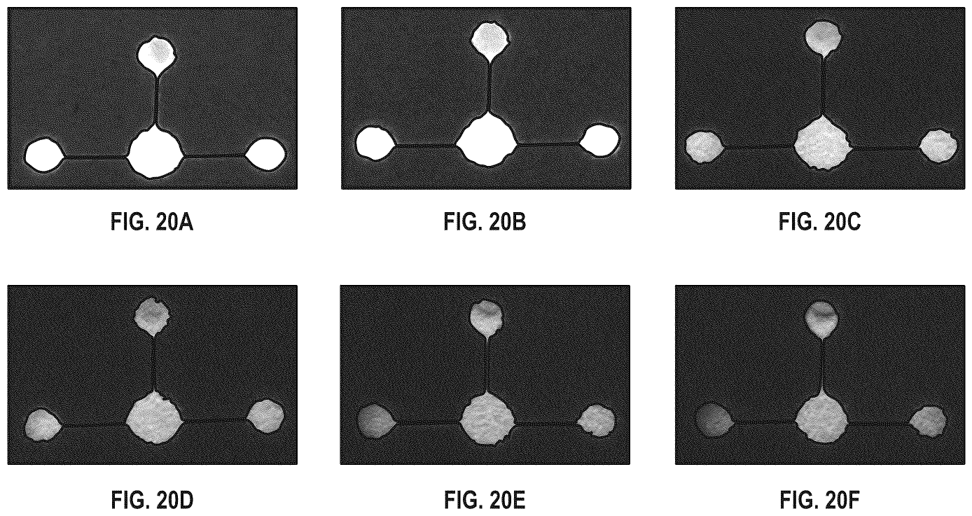

The amount of sample volume containing glucose (8.0 mmol/L), lactate (8.0 mmol/L), and IL-6 (4.0 ng/ml) was measured by introducing the different volumes of sample solution into the sample inlet of the developed μPAD sensor. Experimentally, the suitable sample volume was evaluated by naked eye readout of sensor response. In particular, it was found that there was no color signal change for all the analytes when the sample volume was 2.5 μL, as illustrated in FIG. 20B, which was similar to a blank solution, as illustrated in FIG. 20A. When using a sample volume of 3.5 μL as illustrated in FIG. 20C, a pale-yellow color in the glucose and lactate channels was observed, although the color product did not fully reach their respective detection zones. Similarly, there was no change in the detection zone of IL-6. When the sample volume was 5.0 μL, as illustrated in FIG. 20D. Further, it was found that the apparent yellow color was obtained in the glucose channel while the color signals of lactate and IL-6 channel were not observable. Nevertheless, a yellowish-brown was acquired in the glucose and lactate zone when the sample volume was 7.5 μL, as illustrated in FIG. 20E. At this level, it was found that the distance of the yellow color in the detection zone of IL-6 measurement reduced significantly. Likewise, the color signals in both the glucose and lactate channel increased dramatically, whereas the distance signal of yellow color in the IL-6 channel remain stable when the sample volume was 10.0 μL, as illustrated in FIG. 20F. These phenomena can be discussed in terms of the total mole amount of analyte in the paper microchannel. For colorimetric detection of glucose and lactate, when sample volume increases, it is associated with increasing their moles in concentration on the paper substrate, so they could flow into their specific channel and react with the pre-deposited enzyme to generate H₂O₂. After that, H₂O₂ as a product can oxidize potassium iodide (KI) in the same channel to iodine (I₂), leading to the color change from colorless to dark brown. Conversely, the distance signal of IL-6 was constant when the sample volume increased. Since the mole of IL-6 could not bind to a pre-deposited bromothymol blue (BTB) in the paper fluidic device, the result demonstrated the unchanged distance signal in this zone. Even though the color intensity of glucose and lactate sensing were higher, 7.5 μL of sample volume was selected for the developed μPAD sensor.

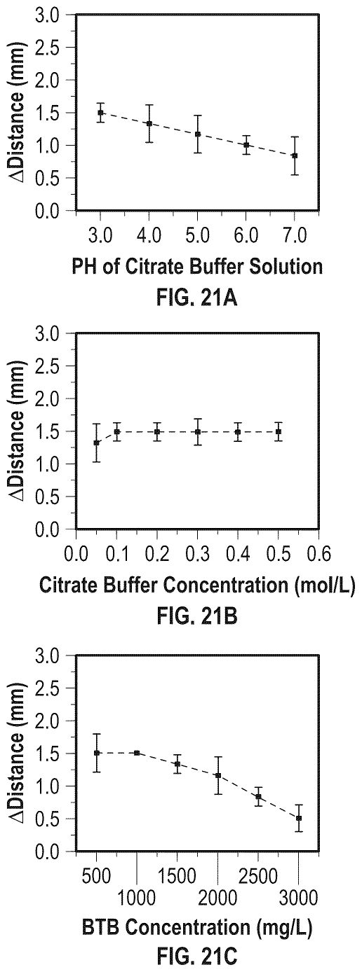

The parameters that can affect the developed method for simultaneous detection of glucose, lactate, and IL-6 are further evaluated by detecting the color intensity for glucose and lactate, and distance length for IL-6. To that end, the conditions for dry-binding protein detection of IL-6 were investigated. The pH of citate buffer solution was studied in particular since it affects the binding ability between the IL-6 and the Bromothymol blue (BTB) molecule, as illustrated in FIG. 21A. It was found that the distance signal was reduced as pH was increased. The isoelectric point (pI) value of IL-6 was 6.2, meaning that a pH of citate buffer solution lower than this value could activate the positive charge on the surface of IL-6. Thus, this positive charge on the IL-6 molecule was able to bind to the negative charge on BTB molecule, leading to a movement of bound product along a distance. However, at a pH value higher than 6.2, the results showed significant decrease in signal with high error in measurement because of the reduction of the positive charge on IL-6 molecule, thus resulting in non-binding with BTB molecule in the system. Overall, a pH of 3.0 was selected as an appropriate pH value in order to enhance detectability of the device. After that, the citate buffer was tested in the ranges from 0.05 to 0.50 mol/L. Referring specifically to FIG. 21B, there was a significant rise of the distance signals as concentrations increased, but distances remained stable when the concentration was higher than 0.10 mol/L. The citate buffer concentration at 0.10 mol/L was chosen to be an adequate level in this example to reduce reagent consumption. Subsequently, the BTB concentration was evaluated since it is a significant contributor to the dry-binding technique. For example, and as illustrated in FIG. 21C, the highest distance signal was obtained when the lowest BCP level was used to coat the paper. The higher level of BTB was affected by the reduction of the binding efficiency in terms of the binding constant (Kc) by Eq. 1, where [BTB-IL-6] as ([BTB-IL-6/]+[BTB-IL-6//]) and [BTB] as ([BTB-]+[BTB2-]):

$${K}_{c} = [ B TB - IL - 6 ] / [ B TB ] [ IL - 6 ] \qquad Eqn . 1$$

However, at lowest BTB of 500.0 mg/L indicated significant tolerance of 0.29. To avoid the error for measurement, the BTB concentration at 1000.0 mg/L was selected as the potential concentration for the present example.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks, e.g., compact disks and digital video disks, magnetic cassettes, memory cards or sticks, random access memories (RAMs), read only memories (ROMs), and the like.

As used in the claims, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

The present disclosure has been described in terms of one or more preferred embodiments, and it should be appreciated that many equivalents, alternatives, variations, and modifications, aside from those expressly stated, are possible and within the scope of the disclosure.

I claim:

1. A sampling system comprising:
a patch including at least one grooved microneedle coupled thereto;
a collection reservoir coupled to the patch; and
a sensor coupled to the collection reservoir,
wherein the at least one grooved microneedle defines a fluid channel therein, and
wherein the collection reservoir is in fluid communication with the fluid channel of the at least one grooved microneedle.

2. The sampling system of claim 1, wherein a first side of the patch includes an adhesive backing.

3. The sampling system of claim 1, wherein the at least one grooved microneedle is one of a plurality of grooved microneedles, and wherein the plurality of grooved microneedles is arranged in an array on the patch.

4. The sampling system of claim 1, wherein the sensor is provided as an absorbent bead that is coupled to the at least one grooved microneedle and retains fluid received from the at least one grooved microneedle.

5. The sampling system of claim 4, wherein the absorbent bead is configured to detect one or more of pH, glucose, lactate, or interleukin-6 in the fluid received from the at least one grooved microneedle.

6. The sampling system of claim 4, wherein the absorbent bead includes a one way valve that allows air to exit the absorbent bead when compressed.

7. The sampling system of claim 4, wherein the absorbent bead expands and creates suction through the at least one grooved microneedle after being compressed.

8. The sampling system of claim 4, wherein the absorbent bead retaining the fluid received from the at least one grooved microneedle is removed the collection reservoir to analyze the fluid.

9. A system for sampling a fluid of a subject, comprising:
a patch including at least one microneedle coupled to a first side thereof; and
a sensor to detect a presence of at least one biomarker in the fluid that is drawn through the at least one microneedle,
wherein the at least one microneedle includes a body with a central channel therein, the central channel being in fluid communication with a groove disposed on an exterior of the body.

10. The system of claim 9, wherein the at least one microneedle defines an axial length between an apex and a base along a longitudinal axis, and wherein the base defines an outer diameter of the microneedle that is less than the axial length.

11. The system of claim 10, wherein the groove defines a groove length along the longitudinal axis, and
wherein the groove length is between 30% and 100% of the axial length of the at least one microneedle, expressed as a percentage.

12. The system of claim 9, further comprising a pump coupled to a second side of the patch, the pump providing a suction force to direct the fluid through the at least one microneedle from a region of interest of the subject.

13. The system of claim 12, wherein the pump is one or more of a micropump, a syringe pump, or a vacuum pump.

14. The system of claim 9, wherein the sensor is a removable cartridge that is coupled to a second side of the patch.

15. The system of claim 9, wherein the sensor is a paper-based sensor that includes a sample zone and at least one detection zone that is in fluid communication with the sample zone.

16. A method of sampling interstitial fluid, the method comprising:
placing, on a subject's skin, a microneedle patch including at least one microneedle coupled a first side of the microneedle patch and a collection reservoir coupled to a second side of the microneedle patch,
providing a suction force to the microneedle patch to direct a fluid through the at least one microneedle and into the collection reservoir; and
detecting, with a sensor coupled to the collection reservoir, at least one of a presence or a concentration of a biomarker in the fluid.

17. The method of claim 16, wherein the suction force is provided by decompressing an absorbent bead coupled to the at least one microneedle.

18. The method of claim 16, wherein the suction force is provided by a pump coupled to the collection reservoir.

19. The method of claim 16, wherein the biomarker comprises one or more of pH, glucose, lactate, or interleukin-6.

20. The method of claim 16, wherein detecting the presence of the biomarker includes:
providing the fluid to a sample zone of the sensor; and
analyzing a color of one or more detection zones of the sensor.
